(19) United States
(12) Patent Application Publication
(10) Pub. No.: US 2021/0321208 A1
(43) Pub. Date: Oct. 14, 2021

(54) PASSIVE FITTING TECHNIQUES

(71) Applicant: Cochlear Limited, Macquarie University, NSW (AU)

(72) Inventor: Toby CUMMING, Macquarie University (AU)

(21) Appl. No.: 17/269,605

(22) PCT Filed: Oct. 25, 2019
(86) PCT No.: PCT/IB2019/059173

§ 371 Date: Feb. 19, 2021

Related U.S. Application Data
(60) Provisional application No. 62/750,394, filed on Oct. 25, 2018.

(51) Int. Cl. H04R 25/00; A61N 1/36

(52) U.S. Cl. CPC H04R 25/70; H04R 2225/43; A61N 1/36039

(57) ABSTRACT

A fitting system, including a communications subsystem including at least one of an input subsystem and an output subsystem or an input/output subsystem, and a processing subsystem, wherein the processing subsystem is configured to automatically develop fitting data for a hearing prosthesis at least partially based on data inputted via the communications subsystem.

### CROSS-REFERENCE TO RELATED APPLICATIONS

This application claims priority to U.S. Provisional Application No. 62/750,394, entitled PASSIVE FITTING TECHNIQUES, filed on Oct. 25, 2018, naming Toby CUMMING of Macquarie University, Australia as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant. Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from hearing loss typically receive an acoustic hearing aid. Conventional hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve. Cases of conductive hearing loss typically are treated by means of bone conduction hearing aids. In contrast to conventional hearing aids, these devices use a mechanical actuator that is coupled to the skull bone to apply the amplified sound. In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound. Many devices, such as medical devices that interface with a recipient, have structural and/or functional features where there is utilitarian value in adjusting such features for an individual recipient. The process by which a device that interfaces with or otherwise is used by the recipient is tailored or customized or otherwise adjusted for the specific needs or specific wants or specific characteristics of the recipient is commonly referred to as fitting. One type of medical device where there is utilitarian value in fitting such to an individual recipient is the above-noted cochlear implant. That said, other types of medical devices, such as other types of hearing prostheses, exist where there is utilitarian value in fitting such to the recipient.

### SUMMARY

In an exemplary embodiment, there is a fitting system, comprising, a communications subsystem including at least one of an input subsystem and an output subsystem or an input/output subsystem, a processing subsystem, wherein the processing subsystem is configured to automatically develop fitting data for a hearing prosthesis at least partially based on data inputted via the communications subsystem.

In an exemplary embodiment, there is a method comprising capturing speech using a machine and automatically developing, based on the captured speech, fitting data for a hearing prosthesis.

In an exemplary embodiment, there is a non-transitory computer-readable media having recorded thereon, a computer program for executing at least a portion of a hearing-prosthesis fitting method, the computer program including code for enabling a obtaining of first data indicative of a speech environment of the recipient, code for analyzing the obtained first data; and code for developing fitting data based on the analyzed first data.

In an exemplary embodiment, there is a method, comprising fitting a sensory prosthesis for a recipient based on at least 750 hours of hearing prosthesis recipient participation obtained within a 9000-hour period.

In an exemplary embodiment, there is a device, comprising a processor; and a memory, wherein the device is configured to receive input indicative of speech sound, the device is configured to analyze the input indicative of speech sound, and identify anomalies in the speech sound based on the analysis of the input, which anomalies are statistically related to hearing prosthesis fitting imperfections.

In an exemplary embodiment, there is a method, comprising capturing speech sound with a body carried device, wherein the speaker is a recipient of the hearing prosthesis, evaluating data, wherein the data is based on the captured speech, and developing fitting data based on the evaluated data and at least one of at least partially fitting or at least partially adjusting a fitting of the hearing prosthesis based entirely on the developed fitting data without an audiologist.

### BRIEF DESCRIPTION OF THE DRAWINGS

Embodiments are described below with reference to the attached drawings, in which:

FIG. 1 is a perspective view of an exemplary hearing prosthesis in which at least some of the teachings detailed herein are applicable;

FIGS. 2A and 2B presents an exemplary system including a hearing prosthesis and a remote device in the form of a portable handheld device;

FIGS. 3, 4 5 and 6 present schematics of exemplary algorithms and systems;

FIGS. 7 and 8 present exemplary functional block diagrams;

FIGS. 9-14 present exemplary flowcharts for exemplary methods; and

FIGS. 15-20 present additional functional diagrams.

### DETAILED DESCRIPTION

Embodiments will be described in terms of a cochlear implant, but it is to be noted that the teachings detailed herein can be applicable to other types of hearing prostheses, and other types of sensory prostheses as well, such as, for example, retinal implants, etc. In an exemplary embodiment of a cochlear implant and an exemplary embodiment of system that utilizes a cochlear implant with remote components will first be described, where the implant and the system can be utilized to implement at least some of the teachings detailed herein.

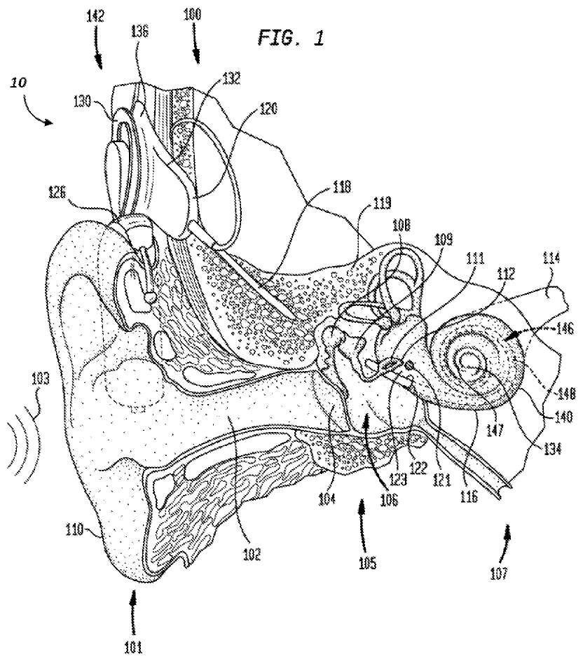

FIG. 1 is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. Additionally, it is noted that the teachings detailed herein are also applicable to other types of hearing prostheses, such as by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing. Accordingly, any disclosure herein with regard to one of these types of hearing prostheses corresponds to a disclosure of another of these types of hearing prostheses or any medical device for that matter, unless otherwise specified, or unless the disclosure thereof is incompatible with a given device based on the current state of technology. Thus, the teachings detailed herein are applicable, in at least some embodiments, to partially implantable and/or totally implantable medical devices that provide a wide range of therapeutic benefits to recipients, patients, or other users, including hearing implants having an implanted microphone, auditory brain stimulators, visual prostheses (e.g., bionic eyes), sensors, etc.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which can be configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

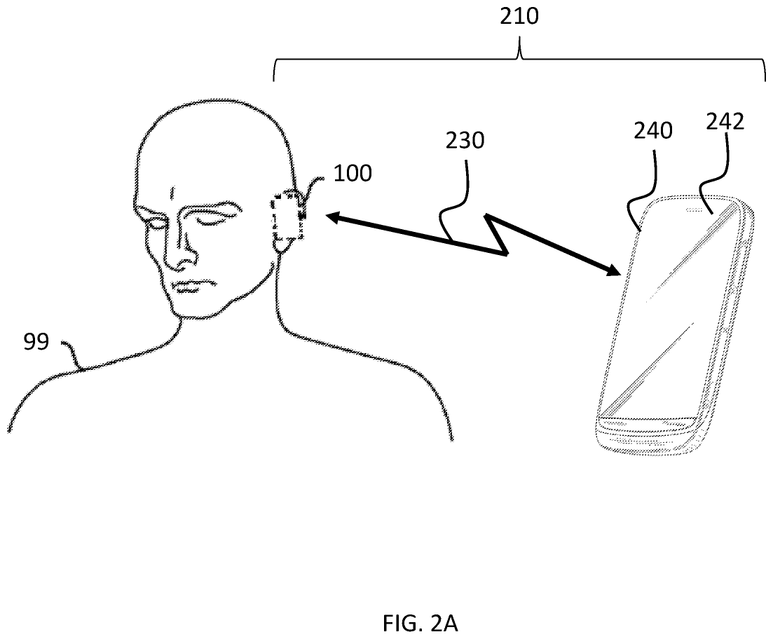

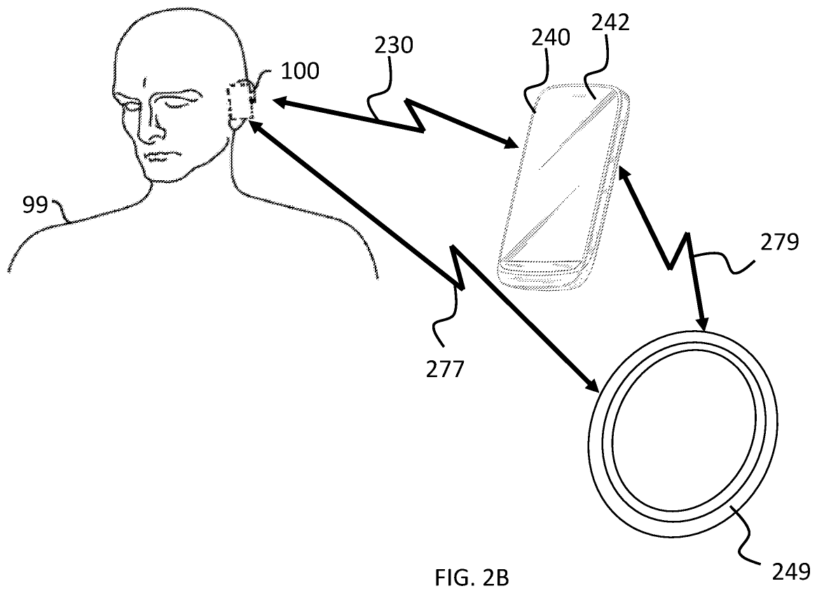

FIG. 2A depicts an exemplary system 210 according to an exemplary embodiment, including hearing prosthesis 100, which, in an exemplary embodiment, corresponds to cochlear implant 100 detailed above, and a portable body carried device (e.g. a portable handheld device as seen in FIG. 2A, a watch, a pocket device, etc.) 240 in the form of a mobile computer having a display 242. The system includes a wireless link 230 between the portable handheld device 240 and the hearing prosthesis 100. In an exemplary embodiment, the hearing prosthesis 100 is an implant implanted in recipient 99 (as represented functionally by the dashed lines of box 100 in FIG. 2A). Again, it is noted that while the embodiments detailed herein will be described in terms of utilization of a cochlear implant, the teachings herein can be applicable to other types of sensory prostheses. Any disclosure of application of the teachings herein to one specific prostheses corresponds to a disclosure of an alternate embodiment where those teachings are applied to another prosthesis listed herein, unless otherwise noted, providing such is enabled.

In an exemplary embodiment, the system 210 is configured such that the hearing prosthesis 100 and the portable handheld device 240 have a symbiotic relationship. In an exemplary embodiment, the symbiotic relationship is the ability to display data relating to, and, in at least some instances, the ability to control, one or more functionalities of the hearing prosthesis 100. In an exemplary embodiment, this can be achieved via the ability of the handheld device 240 to receive data from the hearing prosthesis 100 via the wireless link 230 (although in other exemplary embodiments, other types of links, such as by way of example, a wired link, can be utilized). As will also be detailed below, this can be achieved via communication with a geographically remote device in communication with the hearing prosthesis 100 and/or the portable handheld device 240 via link, such as by way of example only and not by way of limitation, an Internet connection or a cell phone connection. In some such exemplary embodiments, the system 210 can further include the geographically remote apparatus as well. Again, additional examples of this will be described in greater detail below.

As noted above, in an exemplary embodiment, the portable handheld device 240 comprises a mobile computer and a display 242. In an exemplary embodiment, the display 242 is a touchscreen display. In an exemplary embodiment, the portable handheld device 240 also has the functionality of a portable cellular telephone. In this regard, device 240 can be, by way of example only and not by way of limitation, a smart phone as that phrase is utilized generically. That is, in an exemplary embodiment, portable handheld device 240 comprises a smart phone, again as that term is utilized generically.

It is noted that in some other embodiments, the device 240 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 240 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 240 is not a mobile computer, but instead a remote device (remote from the hearing prosthesis 100. Some of these embodiments will be described below).)

In an exemplary embodiment, the portable handheld device 240 is configured to receive data from a hearing prosthesis and present an interface display on the display from among a plurality of different interface displays based on the received data. Exemplary embodiments will sometimes be described in terms of data received from the hearing prosthesis 100. However, it is noted that any disclosure that is also applicable to data sent to the hearing prosthesis from the handheld device 240 is also encompassed by such disclosure, unless otherwise specified or otherwise incompatible with the pertinent technology (and vice versa).

It is noted that in some embodiments, the system 210 is configured such that cochlear implant 100 and the portable device 240 have a relationship. By way of example only and not by way of limitation, in an exemplary embodiment, the relationship is the ability of the device 240 to serve as a remote microphone for the prosthesis 100 via the wireless link 230. Thus, device 240 can be a remote mic. That said, in an alternate embodiment, the device 240 is a stand-alone recording/sound capture device.

It is noted that in at least some exemplary embodiments, the device 240 corresponds to an Apple Watch™ Series 1 or Series 2, as is available in the United States of America for commercial purchase as of Jun. 6, 2018. In an exemplary embodiment, the device 240 corresponds to a Samsung Galaxy Gear™ Gear 2, as is available in the United States of America for commercial purchase as of Jun. 6, 2018. The device is programmed and configured to communicate with the prosthesis and/or to function to enable the teachings detailed herein.

In an exemplary embodiment, a telecommunication infrastructure can be in communication with the hearing prosthesis 100 and/or the device 240. By way of example only and not by way of limitation, a telecoil 249 or some other communication system (Bluetooth, etc.) is used to communicate with the prosthesis and/or the remote device. FIG. 2B depicts an exemplary schematic depicting communication between an external communication system 249 (e.g., a telecoil), and the hearing prosthesis 100 and/or the handheld device 240 by way of links 277 and 279, respectively (note that FIG. 2B depicts two-way communication between the hearing prosthesis 100 and the external audio source 249, and between the handheld device and the external audio source 249—in alternate embodiments, the communication is only one way (e.g., from the external audio source 249 to the respective device)).

Briefly, it is noted that in an exemplary embodiment, various components disclose herein can be all part of a single processor, providing that the art enables such, while in other embodiments, the components herein are separate processors/parts of separate processors. Thus, in an exemplary embodiment, there is a processor or a plurality of processors that are programmed and configured or otherwise contains code or otherwise have access to code (e.g., a memory storing such code, or some firmware or software or hardware, etc.), to execute one or more of the functionalities detailed herein and/or to execute one or more of the method actions detailed herein. Further, in an exemplary embodiment, this processor(s) can include or otherwise be configured to execute the functions/actions herein.

In an exemplary embodiment, the aforementioned processor(s) can be a general-purpose processor(s) that is configured to execute one or more the functionalities/actions herein. In an exemplary embodiment, the aforementioned processor is a modified cochlear implant sound processor that has been modified to execute one or more of the functionalities detailed herein. In an exemplary embodiment, a solid-state circuit is configured to execute one or more of the functionalities/actions detailed herein. Any device, system, and/or method that can enable the teachings detailed herein can be utilized in at least some exemplary embodiments. In an exemplary embodiment, a personal computer or a smart device is programmed to execute the teachings herein.

Some examples of configuring a hearing prosthesis, such as a cochlear implant, include doing so for a recipient relying on a clinician to measure the comfort levels (C-levels), and threshold levels (T-levels) across the electrode array. One or more or all electrodes are mapped to a certain frequency range, and the output, along with other variables that affect the output delivered by the implant (e.g., rate, pulse width, maxima, gains, etc.), is referred to as the patient's ‘MAP’.

Some examples of fitting a hearing prosthesis to a recipient can include measuring C and T levels for each of the electrodes of the array. For example, a 10-electrode array would have 20 measurements, a 16-electrode array would have 32 measurements, a N electrode array would have N×2 measurements (e.g., if N=22 (22 electrodes), there would be 44 measurements). Some examples also include executing objective measures (for example, obtaining Neural Response Telemetry (NRT) levels) and/or interpolation, where a smaller number of T or C levels are measured (for example, 5 threshold levels for the streamlined method). Levels for the intermediate electrodes are calculated or otherwise identified using an algorithm, such as that based in software and loaded onto a computer. This example of MAP development does not indicate how the recipient is handling or otherwise comping with his or her situation. This example of MAP development also does not indicate how the recipient is handling or otherwise coping with the changes in the parameters, etc., which parameters are affecting the recipient's performance. An example of obtaining data that indicates such can be achieved via the implementation of various outcome measures. For example, informal techniques can be executed, such as, for example, executing a ling sound check. Also, for example, outcome questions can be used (e.g., asking the recipient to rate his or her ability to perform certain tasks). Also, for example, performance tests can be used. For example, aided audiograms can be used or otherwise developed, which include detecting the level where the recipient can hear the very softest sound with the aid of the device (in this case a Cochlear Implant). Also, for example, word testing can be executed, where, for example, a set of words can be played or otherwise provided to the recipient and the recipient is then queried (e.g., asked) or instructed to repeat the words. The clinician or other professional then scores which words (or phonemes) the patient has heard correctly.

Further by example, sentence testing can be executed. In an example, recordings of whole sentences are played or otherwise presented to the patient and the recipient is asked or otherwise instructed to repeat the sentences. These sentences may be played or otherwise provided in quiet (with no background noise), or in a non-quite environment (e.g., with background noise).

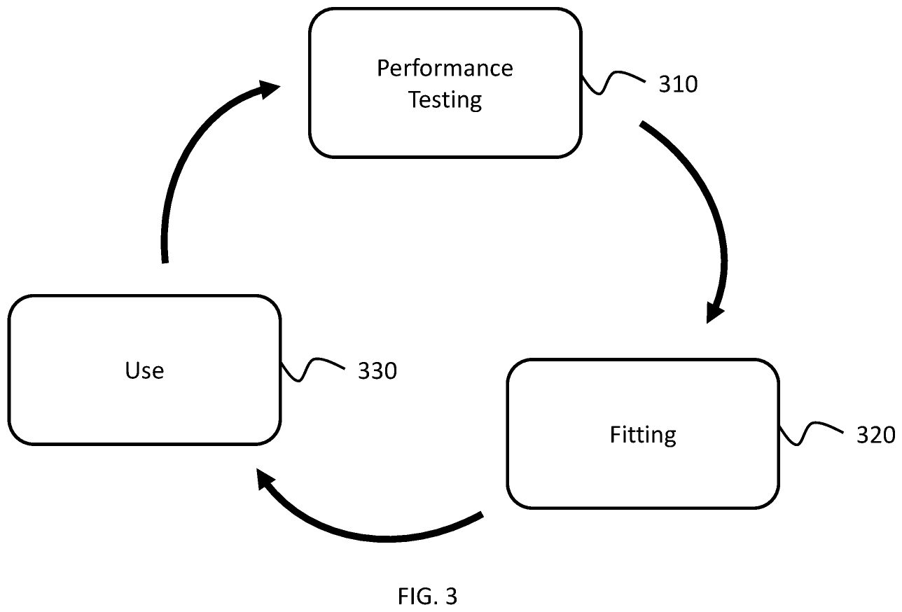

In at least some of the above noted examples, the tests require a specialist setup, such as the use of a soundproof room, calibrated speakers, etc.). A first temporal period is required to execute the tests. FIG. 3 presents an exemplary diagram of an exemplary cycle associated with the teachings detailed above. In the diagram presented on FIG. 3, there is method action 310, which entails performance testing, method action 320, which entails fitting, and method action 330, which entails the utilization of the prosthesis. In at least some exemplary embodiments associated with the methods associated with the diagram of FIG. 3, the testing and/or the fitting steps can occur in the clinic. Still further by way of example only and not by way of limitation, in an exemplary embodiment, the utilization method portion of the cycle, method action 330, is executed outside the clinic and/or is not a “task” as such.

In some examples, a testing, fitting, use cycle may then be followed, where the outputs of testing are used, directly and/or indirectly, to inform the fitting that the clinician follows. In at least some exemplary embodiments, the 2 activities are not directly connected. For example, the testing can come after the fitting as a validation of the updates that were made to the prosthesis and/or the programming/mapping associated there with. Alternatively, the testing may be used to track overall progress rather than inform specific MAP updates. In at least some examples, both testing and fitting are time-intensive tasks, so testing may not be performed at every session. Also, in some examples, fitting is a subjective task, and thus one patient visiting a plurality of different clinicians may end up with respective different maps, which in some examples, are very different from each other. In an exemplary embodiment, this can be done by automatically using the outputs from testing to make map adjustments.

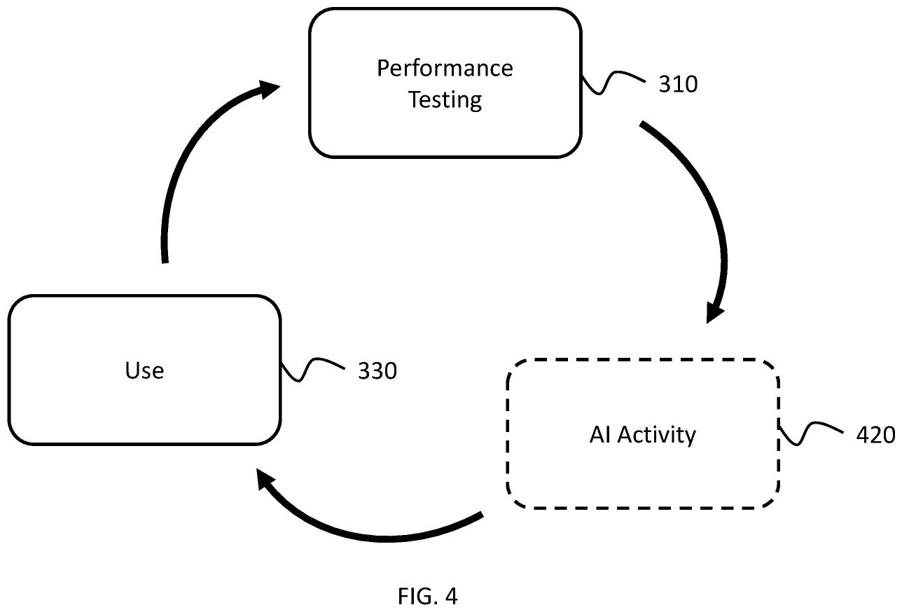

An exemplary embodiment includes an AI (Artificial Intelligence) or expert rules-based system or otherwise some form of machine learning system that is utilized to remove or otherwise reduce the value of and/or the impact of fitting. Briefly, FIG. 4 presents an exemplary diagram depicting a cycle that utilizes AI. Here, there is method action 420, which includes the implementation of artificial intelligence activity. In an exemplary embodiment, the AI activity of method action 420 is artificial intelligence fitting. In this exemplary embodiment, the fitting is removed as a clinical step, and the fitting happens automatically based on the outputs of the testing. This is why the box in the diagram of FIG. 4 for method action 420 is dashed, as it does not represent a task per se but instead represents an action that is somewhat seamless with respect to the other two actions.

Again, in some exemplary embodiments of method action 420, the AI activity can be fitting the hearing prosthesis, wherein alternatively and/or in addition to this, in other exemplary embodiments, the AI activity is identifying issues associated with the data. Still further, in an exemplary embodiment, the AI activity can correspond to developing fitting data, which is separate from applying that data to a prosthesis.

In some exemplary scenarios of use, this can result in less time being required in clinic and/or could remove some of the variability seen in the fitting process. Further, with respect to the facet that there is the issue that the clinician and recipient need to spend significant amounts of time in the clinic running the tests in at least some examples, it is possible that in some exemplary embodiments, one or more or all of the tests may be run at home or otherwise at a location away from the clinic and/or without the clinician involve. In some exemplary embodiments of such, the recipient still must spend significant amount of time running the tests.

It is noted that some examples exist where if a recipient of a hearing prostheses is with a clinician or other professional with understanding of the recipient-prostheses interaction relationship, for a utilitarian amount of time, or otherwise for a significant amount of time, the clinician or the professional may, in some instances, notice some issues just by observation (as opposed to testing). To varying extents this information could be used to inform the clinician's approach to mapping. By way of example only, the clinician may notice the recipient has trouble distinguishing between ss and sh sounds, and may choose to focus on high-frequency sounds in the next MAPing session. For example, by perhaps boosting thresholds in this area.

Accordingly, in an exemplary embodiment, there exists a system that can utilize AI technologies to automatically detect issues and identify or otherwise recommend corrective adjustments to the recipient's map. In an exemplary embodiment, the artificial intelligence duplicates or otherwise replicates or otherwise provides something analogous to that which would result if a human being was listening in to the conversations of the recipient and/or making judgments as to the issues associated with the recipient and his or her hearing prostheses and identifying or otherwise recommending adjustments accordingly. In some exemplary embodiments, such can be achieved by utilizing the processing power available in a recipient's home and/or even with the recipient's hearing prostheses, to implement AI technologies.

Briefly, embodiments can include a cochlear implant sound processor or other component of a cochlear implant system (or a component of another type of hearing prosthesis—again, embodiments are not limited to cochlear implants, but are applicable to any type of hearing prostheses or any other type of sensory prostheses to which the teachings detailed herein can have utilitarian value), where there exists an ability to stream audio content to a phone, such as smart phone 240, or to any other device that can receive such streamed audio. In an exemplary embodiment, this can enable access to the increased processing power available on modern smart phones or the like. That said, in an exemplary embodiment, the smart phone or other remote device to which the data is streamed can be any device that can record and otherwise preserve the audio data or data indicative or otherwise based on the audio content of that stream data, so that it can be later analyzed by another device, which device can have more processing power or otherwise be provided with the algorithms that enable the teachings detailed herein.

Embodiments can also include the utilization of devices and/or systems associated with otherwise that can enable speech/voice recognition. By way of example only and not by way of limitation, such can be present in smart phones or other personal handheld or body carried devices, and in some embodiments, can be located in the hearing prostheses or be part of the hearing prostheses. In an exemplary embodiment, such can enable artificial intelligence to understand the content of what is being said or otherwise extrapolate utilitarian features there from which can be used to implement some of the teachings herein.

Still further, some exemplary embodiments include the utilization of artificial intelligence systems that have the ability to understand or otherwise extrapolate the context of conversations. Moreover, in some exemplary embodiments, there is the utilization of own voice detection technologies. In an exemplary embodiment, these own voice detection technologies are implemented in the hearing prostheses. Any device, system and/or method that can enable own voice detection to implement can be utilized in some exemplary embodiments. Still further, own voice detection can be utilized with non-hearing prostheses devices. In an exemplary embodiment, own voice technologies can be implemented in smart phones or the like or any other suitable device. In an exemplary embodiment, this can enable the determination of the difference between the recipient's own voice and the voices of other people. This can have utilitarian value with respect to having the artificial intelligence system or identifying to the AI system who is speaking, where the AI system can utilize that in its analysis.

Embodiments can include an artificial intelligence system that passively “listens in” on a recipient as he or she goes about their daily lives. That said, embodiments can include a system that enables the provision of data indicative of what would occur if the AI system “listens in” on the recipient, which data is then provided to the AI system. In an exemplary embodiment, this can be a recording of the sounds associated with the recipient of the prosthesis, which recording could be provided to the AI system every night before the recipient goes to bed or per week, etc.

In an exemplary embodiment, the data provided to the AI system that is indicative of the data that would result from a device that is “listening in” is utilized by the AI system to determine for example, whether there are any problems or otherwise abnormalities or issues with the patient's detection of certain sounds, ability to discriminate between sounds, or comprehension of what is being said. In an exemplary embodiment, once detected, these issues can be addressed by making changes to the recipient, or at least recommending changes to the recipient's map. In an exemplary method, the system would then monitor the patient's real-world performance (again, via the AI system listening in in real time, or by providing recordings periodically, etc., to the AI system) to determine if the changes improved overall performance and/or limited performance. Such a system can be, in some exemplary embodiments, fully automatic, some embodiments can require no intervention from the recipient, and some embodiments can be systems where the system asks or otherwise needs permission from the recipient to apply an optimized or improved map setting or make adjustments to the prosthesis.

FIG. 5 presents an exemplary diagram according to an exemplary embodiment. Here, FIG. 5 corresponds to FIG. 4, except that method action 310 is replaced with method action 510, which corresponds to performance monitoring. Concomitant with the scheme implemented in FIG. 4, the box for method action 510 is dashed to indicate that this is a non-task action. Again, in some exemplary embodiments of method action 420, the AI activity can be fitting the hearing prosthesis, wherein alternatively and/or in addition to this, in other exemplary embodiments, the AI activity is identifying issues associated with the data. Still further, in an exemplary embodiment, the AI activity can correspond to developing fitting data, which is separate from applying that data to a prosthesis.

In an exemplary embodiment, the cycle of FIG. 5 can be repeated over and over ABC number of times, where ABC can equal 1, 2, 3, 4, 5, 6, 7, 8, 9, 10, 11, 12, 13, 14, 15, 16, 17, 19, 20, 21, 22, 23, 24, 25, 26, 27, 28, 29, 30, 35, 40, 45, 50, 55, 60, 65, 70, 80, 90, 100, 110, 120, 130, 140, 150, 160, 170, 180, 190, 200, 225, 250, 275, 300, 350, 400, 450, 500, 600, 700, 800, 900, or 1000 or more or any value or range of values therebetween in integer increments. In an exemplary embodiment, the limiting factor with respect to the number of times that the cycle of FIG. 5 can be repeated could be the fatigue of the recipient with respect to any need for the recipient to reactivate himself or herself to the new maps that are developed.

That said, because the system is configured to execute at least sometimes without input from the recipient (and in other times, the recipient can provide input if he or she wants to, but such is not necessarily needed for the cycles to be implemented), the system could be relatively transparent to the recipient, and the recipient may or may not notice the changes to the maps with each cycle. Indeed, in an exemplary embodiment, an incremental approach could be taken where the system identifies a change to the map that could be utilitarian, but the map is not outrightly changed to that ultimate change. Instead, an incrementalist approach is applied where the change is made to the map, which change may not necessarily fully address a problem identified with the mapping, but is made in a manner that is not entirely noticeable if at all by the recipient. The system could change the map settings over the course of hours or days or even weeks to arrive at the desired change, in the meantime acclimating the recipient to each subtle change so that the ultimate change is somewhat transparent if not totally transparent to the recipient (other than the ultimate result of being able to better hear).

In an exemplary embodiment of the methods associated with the diagram of FIG. 5, performance testing in the clinic is replaced by passive performance monitoring in the field and/or by passive performance data acquisition in the field. As will be detailed below, the cycle of FIG. 5 can also include, periodically or in some instances, active performance monitoring in the clinic or remote from the clinic. By way of example only and not by way of limitation, the cycle could be repeated 5, 10, 15, 20, 25 or 30 times before active performance monitoring is executed, and then however many cycles could be repeated based solely on passive performance monitoring, until another active performance monitoring event takes place, and so on.

FIG. 6 presents another exemplary diagram representing another exemplary method. As can be seen, there is the action of speech generation, represented by action 610. This speech is captured at method action 620 via performance monitoring. The method also includes method action 630, which includes automatic map revision identification, followed by method action 640, which corresponds to map changes based on method action 630. Also as can be seen, there is the action of the occurrence of anomalies in block 698 and the identification of such anomalies as errors in block 699 (or non-errors—as will be detailed herein, anomalies do not always equate to errors, and this is a feature of the teachings detailed herein that can have further utilitarian value as opposed to embodiments that do not have this feature). That said, in some alternate embodiments, all anomalies are considered errors.

Still, with respect to the error detection process, in an exemplary embodiment, the teachings detailed herein can be utilized to detect and otherwise define an error or a mistake from the pool of anomalies. Of course, corollary to this is that at least some exemplary embodiments also enable the identification of anomalies in the first instance. In this regard, an anomaly is a genus and errors are a species of anomalies. All errors are thus anomalies, but not vice versa. The teachings detailed herein can be utilized to distinguish between the two or otherwise identify errors from the overall anomalies.

As a baseline, it is to be understood that speech testing can generally easily provide a definition of what is correct and/or what is considered a mistake. The issue here is that the recipient is not being set a test or is not undergoing testing during the action of speech generation. Thus, the error detection and testing paradigm cannot be implemented, at least not directly, into the system to detect mistakes.

Consider further that as a baseline, if a clinician or other trained professional is with a recipient for a utilitarian amount of time or an effective amount of time, that person may begin to notice some issue. In some instances, this is simply and/or totally by observation. Teachings detailed herein utilize and AI system that can notice errors, including subtle errors, at least as well as a human could notice such, and in some embodiments, better than a human could notice such. Indeed, in an exemplary embodiment, the noticed errors noticed by the AI system are even more subtle and much more subtle than that which would be noticeable by a human listener.

In an exemplary embodiment, the AI system can identify/classify errors utilizing, as a framework, the various levels of hearing skill, such as so-called detection, discrimination, identification and comprehension. In an exemplary embodiment, detection can be implemented by identifying using the AI system the occurrence of a sound, which sound is deemed to be one that the recipient should have heard (an exemplary embodiment includes automatic determination of this, which distinguishes from other sounds), and identification that the listener did not respond. In an exemplary embodiment, this can be identified as an anomaly. In an exemplary embodiment, the AI system can evaluate the level of the sound. If the level of the sound is sufficiently high that it is unlikely as a statistical manner that the recipient did not hear the sound, the system might discount this anomaly and identify it not as an error because the possible cause could be there if you just did not want to answer. Indeed, in an exemplary embodiment, the system can utilize voice identification to catalog people that the recipient tends to ignore for whatever reason. That said, in an exemplary embodiment, if the sound is of a low-level magnitude that it is plausible that the recipient did not hear the sound, the system could identify the lack of a response as an error. Note that in some embodiments, all lack of response could be identified as error providing that the system determines that the sound was a sound that should have elicited a response in the first instance. Note also that in some embodiments, the system can determine that a lack of response was in fact due to the recipient not hearing, but can factor in the scenario where the recipient should not have heard based on a normal hearing listener. That is, if someone spoke so softly that a normal hearing listener would not be able to hear that person (or might just be inclined to ignore that person because there speaking in a manner that might warrant the person being ignored so as to avoid moral hazard), the system might indicate that the anomaly is not an error.

Further, in an exemplary embodiment, the system could be aware of the listening stage of the recipient and adjust the algorithm's expectations accordingly or otherwise adjust the output accordingly. The determination of the listening stage can be based on a latent variable if you will, or otherwise tangential data that is statistically correlated to the stage. For example, as a child grows up they will move through the stages from detection, discrimination identification and comprehension. Based on the given age of the child, the stage of hearing can be estimated. Alternatively and/or in addition to this, analysis of the recipient's performance can be utilized to determine the stage. A combination of latent variables and direct data can be utilized. Any device, system and/or method that can enable the recipients listening stage to be determined or otherwise estimated can be utilized in at least some exemplary embodiments. Thus, in an exemplary embodiment, the latent variables and where the direct variables can be put into the system so that the system will “know” the stage of the recipient. Further, in an exemplary embodiment, the ultimate determination—the stage—can be inputted into the system. The system could also evaluate the data and determine whether or not the inputted stage is correct or otherwise there should be an adjustment.

There are analogies to the progress of a child when it comes to a new cochlear implant recipient, even with respect to an adult. For example, the statistical based expectations for the recipient's abilities with a cochlear implant in week 1 should be very different from the expectations of their abilities at week 52, and week 52 should be different than week 104, etc. There can be correlation between the stages and the amount of time that a recipient has utilized the cochlear implant. Thus, the stages could be predetermined based on age and/or experience using the system and/or could also be based on the observed speech and responses, or any other data set that can have utilitarian value with respect to determining the stage of the recipient. Thus, depending on the stage of the recipient, the output can be varied based thereon. In this regard, performance metrics can be considered/evaluated with respect to the stage of the recipient, and adjustments can be made accordingly. Thus, adjustments to maps/new maps/new training regimes/changes to training regimes can be different if the recipient is at the detection stage vs. the discrimination stage vs. the identification stage vs. the comprehension stage, etc. Changes that might be made for one stage/errors that would be deemed such for one stage may not be done for recipients at other stages. Typically, the higher the stage, the more aggressive the change/the less “tolerant” the system will be to errors, although in other embodiments, this may not be the case (the system can take into account, in some embodiments, “apathy” or “weariness,” where a sophisticated recipient “just does not care,” and thus a level of “sloppiness” may be more tolerated/disregarded).

In an exemplary embodiment, the AI system can identify/classify errors utilizing, as a framework, the various levels of hearing skill, such as so-called detection, discrimination, identification and comprehension. In an exemplary embodiment, detection can be implemented by identifying using the AI system the occurrence of a sound, which sound is deemed to be one that the recipient should have heard (an exemplary embodiment includes automatic determination of this, which distinguishes from other sounds), and identification that the listener did not respond.

In an exemplary embodiment, the system can determine the frequencies of the sound associated with the error.

It is noted that in an exemplary embodiment, the system could evaluate other types of latent variables that can be indicative of whether or not detection occurred. By way of example only and not by way of limitation, if the sound that was captured was coupled with data that indicated a direction from where the sound came from relative to the recipient, the occurrence of a head turning or lack of a head turning in that direction could indicate whether or not the recipient detected that sound.

In an exemplary embodiment, the system could identify issues such as non-responses to certain sounds, such as, for example, phones, alarms, etc., and/or mistakes in response to certain phrases. Depending on the situation, the system could identify this is an error. Note that the data provided to the system could be multifaceted. By way of example only and not by way of limitation, with respect to the embodiment of the phone ringing, the system might be able to determine that the recipient looked at his or her phone or otherwise took actions indicative of the recipient recognizing that the phone was ringing, and thus otherwise just chose not to answer the phone for example. In an exemplary embodiment, the recipient might generate an utterance associated with dismissal of the sound of the like, which would indicate that the recipient did indeed hear the sound but did not take the action that the system otherwise would have expected. Any data set that can enable error determination and/or classification can be utilized in at least some exemplary embodiments.

The system can be configured to evaluate the data with respect to discrimination features. In this regard, by way of example, the system can evaluate the sound and identify differences between sounds, such as, for example, “sh,” and “ss” and/or “ah” and “oo.” Difficulties in discrimination can surface in everyday speech. In an exemplary embodiment, this can be a result of a mistake in a response, such as for example, misunderstanding “shake” for “sake,” and/or the recipient responding inappropriately in a conversation and/or the recipient simply asking the other party to repeat a given phrase, etc. In an exemplary embodiment, the system can be configured to evaluate the data to identify any of these occurrences, and upon the identification of such, identify such as an error, with or without further evaluation.

In some embodiments, the system can be configured to execute the analysis associated with the identification level of hearing. In an exemplary embodiment, this can correspond to the system evaluating the sound to indicate or otherwise determine whether or not the recipient has the ability to label a word and/or a sound. In an exemplary embodiment, a problem with identification or otherwise an error in identification, such as, for example, not recognizing all the words in a sentence, can result in, some embodiments, the meaning of the sentence being lost and/or diluted. In this regard, the system can be configured to evaluate the data and determine whether or not a response is indicative of a recipient who does not understand the full meaning or even any of the meaning of statements made to him or her.

Also, the system can be configured to evaluate the data for features associated with comprehension. In this regard, the system can evaluate whether or not the listener understands the context and/or the meaning of other talkers around him or her. In an exemplary scenario where testing occurs, as opposed to the method associated with FIG. 6, this can be assessed, for example, by asking “what is the next day of the week after Thursday?” If the recipient does not respond “Friday,” this can be indicative of the fact that the recipient did not comprehend the sentence. It is noted that in an exemplary embodiment, comprehension can be difficult when cognitive load is high, such as, for example, when talking on the phone and/or in a noisy location, such as a noisy restaurant.

In at least some exemplary embodiments, the system is configured to evaluate the data that is obtained to identify or otherwise categorize the data as having, by way of example only and not by way of limitation, one or more of the following occurrences: nonresponse, requests for the speaker to repeat and/or clarify and/or inappropriate responses to conversations.

In an exemplary embodiment, nonresponses can be categorized or otherwise identified based on the occurrence of a scenario where the recipient does not respond to a sound, again, such as for example, a phone and/or an alarm. In another exemplary scenario, nonresponse can be identified in a scenario where the recipient does not respond to speech when a response is anticipated by the system. In an exemplary embodiment, the AI system could be configured to evaluate the sound and upon a determination of a nonresponse, determine that there is a problem with respect to detection. In an exemplary embodiment, the AI system could provide an indication of such, such as a report containing the summary of the sound, the time of the occurrence, the evaluation of the sound scene (noisy environment, conversation, background music, etc.) and/or other data, such as location if the system is configured to receive data indicative of such, and/or as well as settings associated with the prostheses at and around the time of the error. In an exemplary embodiment, the AI system can provide a recommended change in the map to the recipient and/or automatically change the map, such changes could entail adjustment of threshold levels for one or more the frequencies associated with the sound that related to the error.

With respect to an exemplary embodiment that evaluates or otherwise determines requests for clarification etc., by way of example only and not by way of limitation, the system can have a predetermined set of keywords such as, by way of example only and not by way of limitation, “excuse me,” “pardon,” sorry,” “eh,” “say again,” “what,” etc., which can be utilized as markers for moments when the recipient does not understand a full sentence or the like and/or asks for repetition or clarification. It is noted that in an exemplary embodiment, the system can be configured to evaluate the features associated with the phrase (frequency, and change of frequency as the word is said, context of the word, etc.) to evaluate whether or not the statement was made in the form of a question or a command, as opposed to another use of the word. For example, the word “sorry” could be evaluated to determine whether or not the frequency increases at the end of the word, indicating a question as opposed to a statement indicative of general apology. Alternatively, and/or in addition to this, the system could evaluate the context with which the word is used. For example, if the word is used in a longer sentence, the system might indicate or otherwise determine that this is not an indication of a problem with the recipient hearing. Alternatively, and/or in addition to this, the system could evaluate the words that were stated before the statement “sorry” was uttered, and evaluate whether or not that is just a general response to the statement precedent.

It is noted that in at least some exemplary embodiments, one or two requests for repetition does not identify the source of the problem. Further, if the speaker asks, “have you seen the latest movie about dogs?” and the patient asks for repetition, the system doesn't know which part of the sentence was not understood. In an exemplary scenario of implementation of the system, the system could develop a log of such problems, and the system could look for patterns in the types of inputs the recipient is having problems interpreting.

Still further, in an exemplary embodiment of where the system evaluates the data to identify an inappropriate response in a conversation, the AI system could evaluate the data and infer anomalies if inappropriate responses are given. This could indicate that the recipient did not understand the meaning of a sentence or the like. This can be analogous to a live speech test, except that there is no test that is set. The real world is the analogous aspect to the test. Thus, in an exemplary embodiment, the data is analyzed to retroactively establish a test, and then the data is utilized to evaluate how well the recipient performed on this retroactive test. Again, in an exemplary scenario, it may be the case that the source of the problem will not be immediately apparent, and thus in an exemplary scenario, the AI system builds up a list of such problems to identify common patterns, etc.

It is noted that the above indicates features that are errors, but are likewise also anomalies. As noted above, there is utilitarian value with respect to differentiating between errors that are indicative of features associated with hearing and other anomalies. In this regard, it is noted that as a threshold matter, making one or more mistakes in a conversation is not uncommon even for those with no hearing difficulty. Indeed, this can frequently occur in challenging listening situations. Problems can also occur to those people without hearing difficulties due to problems with the input. By way of example, poor quality telephone calls, very quiet speech and/or high levels of background noise, all can combine to create anomalies for even someone with the best of hearing. Having the system react to every anomaly that is detected could result in constant changes being applied to the map, and/or the scenario where the user of the output of the system is overwhelmed with information. In an exemplary embodiment, this could become confusing for the recipient and/or the person evaluating the output of the system. Moreover, this could result in the recipient having to frequently adapt and/or re-adapt to these frequent changes to the map. That is, while map changes are utilitarian, the recipient still must adapt to these new changes, and thus there is a fatigue level that can result there from. In any event, such is a waste of time and resources irrespective of the fatigue level of the recipient, and can also result in a perfectly good map setting being changed for no utilitarian reason.

Accordingly, in an exemplary embodiment, any given anomaly can be monitored or otherwise evaluated for frequency or number of occurrences, etc., before a pattern is detected or otherwise determined and the anomaly category is identified as an actionable error. In an exemplary scenario, the system could simply tally the number of anomalies, and upon reaching a certain threshold, in some exemplary embodiments based on a time frame or some other measure (number of words spoken/heard, etc.), the anomaly could then transition to error status.

In an exemplary embodiment, a probabilistic error detection algorithm could be utilized. Note further that in an exemplary embodiment, inputs or feedback could be requested about a given anomaly. In an exemplary embodiment, such as at the end of the day, the system could provide a recipient and/or caregiver a list of anomalies, and the recipient and/or caregiver could identify such as an error or something that should be disregarded. In an exemplary embodiment, this could occur in real time or near real time for that matter.

Below is an exemplary chart for purposes of discussion:

| Anomaly | Anomaly Example | Tally |
|---|---|---|
| /s/ and /sh/ | Shake and sake | I |
| /i/ and /e/ | pin and pen | III |
| /a/ and /o/ | Shark and shock | IIII |

By way of example only and not by way of limitation, in an exemplary scenario, there is every day speech where talkers may not understand everything each other are saying, and this may not have anything to do with hearing difficulties. There may be misunderstandings, requests for clarification (“anomalies” in this context) for one or more different reasons. Background noise levels may be high (e.g., a noisy restaurant), the signal quality may be poor (e.g., a conference call where the speaker is not near the microphone) and/or the listener may just not know the word being used. Any or all of these causes can cause anomalies. However, if the listener has an underlying problem distinguishing between one phoneme and another, then a pattern of such anomalies will emerge, and this will be detectable by the AI system. By way of example only, in effect, the error detection process is like a long drawn out speech test, where the success/fail criteria as the error patterns that are established over time.

Moreover, in an exemplary embodiment, the recipient or other caregiver can provide data in real time to the overall system that is capturing the sound for data to indicate whether or not the data should be used for purposes of identifying errors. By way of example only and not by way of limitation, if a recipient is speaking to a person with a thick accent and/or is speaking to a person who is notorious for not being able to explain himself or herself, the recipient might provide input at the beginning and/or during a conversation indicating that the data should be disregarded. Indeed, in a relatively straightforward example, the recipient could deactivate the recording device during the temporal period associated with the conversation. In an exemplary embodiment, the recipient could then again activate the recording device, and/or the system could be configured to determine that a new conversation has taken place (e.g., by utilizing voice detection techniques where a given voice is no longer present, and thus the system determines or otherwise decides that the phenomenon associated with the recipient not wanting data to impact or otherwise be used by the system has ceased). In any event, any device, system, and/or method that will enable the recipient or caregiver to provide data into the system that will enable the system to discount data or otherwise prevent the system from even obtaining the data for evaluation in the first instance can be used in some embodiments.

Note also that corollary to this, in an exemplary embodiment, there can be an arrangement where the recipient inputs information into the system indicative of the fact that the following data or the data collection or the data that is collected should be evaluated by the system. By way of example only and not by way of limitation, a scenario where the speaker is a person to whom there is some form of attachment (parent-child, employer employee, serious attraction, etc.) could possibly want the map system to be adjusted so that any issues associated with communicating with that person are resolved, even at the expense of resolving other issues.

Thus, it can be seen that in some exemplary embodiments, the system is configured to enable the recipient or caregiver or someone to prioritize the data. In this manner, depending on the prioritization of the data, the system could be more sensitive or less sensitive to treating anomalies as errors.

In any event, once the system has determined that a threshold has been met such that an anomaly can be defined as an actionable error, these mistakes can be categorized or otherwise correlated and a report can be provided to the recipient or caregiver or healthcare professional, etc., for evaluation.

Further, again, once the system has determined that a threshold has been met, in an exemplary embodiment, the mistakes can be fed into a map development framework that can, in at least some exemplary embodiments, improve and/or optimize a given map.

In an exemplary embodiment, the inputs are errors that the AI has identified as such or otherwise as being actionable, and the outputs are MAP parameter changes. In an exemplary embodiment, by way of example, these can include changes to the T and/or C levels, or any other parameters that affect the recipient's hearing, such as by way of example only and not by way of limitation, Q values, frequency allocation, gain, etc.). In an exemplary embodiment, the system is configured such that, as mistakes are noticed and/or map parameters are applied, the “success” of a given change can be determined or otherwise evaluated by monitoring for similar or otherwise relevant mistakes after the map is changed. In an exemplary embodiment, this can be an ongoing process such that whenever the recipient is in a conversation (or a relevant conversation), the number of inputs and/or outputs could grow rapidly in order to train the artificial intelligence system. (Some details of the training of the system are discussed below.)

In an exemplary embodiment, the AI system can be also configured such that it takes objective measures into account in performing the evaluation. By way of example only and not by way of limitation, impedance measurements and/or auto-NRT data can be utilized as inputs, so device characteristics and the recipient's own physiology can be taken into account in developing the map data. Certain changes to the MAP require a period of acclimatization. Thus, in some exemplary embodiments, the determination of whether changes were successful or not may be delayed to take this acclimatization period into account.

An exemplary scenario of use will now be described by way of example only and not by way of limitation. As will be detailed below, any disclosure herein of any method action corresponds to a device and/or a system that is configured to execute such method action providing that the art enables such, unless otherwise noted.

Initially, there can be a first session, where the recipient can be switched on utilizing conventional methods. In an exemplary embodiment, the clinician can run impedance measurements, and/or can run an autoNRT to create or otherwise obtain baseline information and/or can raise the C and T levels to an audible level. In an exemplary embodiment, the results of this first session or that the recipient has access to sound. In an exemplary embodiment, this first session can occur days or weeks or longer after completion of the surgery in which the prosthesis was implanted in the person. In an exemplary embodiment, this first session can occur a few days after the surgery, one week, two weeks, three weeks, four weeks, five weeks, six weeks, seven weeks, eight weeks, nine weeks, or 10 weeks, or later after the implantation surgery is completed. Times could be shorter or longer.

As will be detailed below, exemplary embodiments also include utilizing a trained artificial intelligence system and/or training in artificial intelligence system. In an exemplary embodiment, the results of an NRT test or any other such testing can be utilized for demographic purposes and/or physiological purposes. In an exemplary embodiment, a trained system could have utilitarian value or otherwise have more utilitarian value with respect to recipients that have NRT results than those that are similarly situated, whereas another trained system can have utilitarian value or otherwise have more utilitarian value with respect to recipients having different NRT results than those of the former. By way of example only and not by way of limitation, patients with statistically similar physiology's and were statistically similar demographics may require or otherwise may avail themselves with greater likelihood of success to certain interventions or otherwise therapy regimes relative to other demographic and/or physiological groups. In an exemplary embodiment, age, gender, time of onset of deafness, whether or not the recipient has ever heard naturally, education, work experience, etc., can all impact how data is treated and/or what type of therapies should be utilized. Further, other variables such as skin flap thickness, placement of electrodes in the cochlea and/or outside the cochlea, cause of deafness, etc., can influence the intervention or the therapy based on statistical models. These are some of the demographic inputs and/or physiological inputs that can be used when training the system and/or which can be used for a given system when evaluating what is to be done with respect to the given input. With regard to the latter, a change that would be utilitarian for one group of the population having given NRT results might not be utilized for another group of the population having different NRT results. The trained artificial intelligence system could recognize the physiological inputs and/or the demographic inputs and thus provide output accordingly. It is also noted that the problems or otherwise lack of performance that a four-year-old might experience may be similar to those that are experienced by an 18-year-old were 30-year-old or a 40-year-old, etc. However, it would be reasonable to expect the four-year-old to have these problems and or more of these problems and/or have these problems for a longer period relative to the other age groups just detailed. Accordingly, the changes to a map or therapy, etc. that might be implemented for an older recipient may not be implemented for the younger recipient, at least with respect to a temporal period that is the same with respect to how long the problems persist. The idea is that demographics and/or physiological features can also be included in the mix of variables with respect to determining what is done for a given recipient and/or when it is done, etc. Thus, in some embodiments, any one or more or all of the just detailed variables can be further taken into account or otherwise utilized by the system to develop the new maps or otherwise make a map adjustment, etc. In an exemplary embodiment, only demographic aspects are utilized, while in other embodiments, only physiological aspects are utilized. That said, it is noted that there can be a crossover between the two groups in some embodiments.

Over the first one or two or three or four or five or six weeks after the first session, the recipient tolerates higher levels of electrical stimulation. As the brain begins to adapt to the new inputs provided by the implant, the recipient will develop the ability to comprehend sounds as language. Overall loudness in this early stage can be a simple matter of the recipient bringing up the overall levels. At this early stage, the recipient can be provided with in-person clinician care to assist with the habilitation and/or rehabilitation.

At some point after the initial first session, in an exemplary embodiment, the system is utilized to record or otherwise capture sound associated with a recipient, such as, for example, speech of the recipient and/or speech of people speaking to the recipient or otherwise speech of people around the recipient. This captured sound can correspond to data which is fed in real time, or in increments, or periodically to the AI system. Again, in some embodiments, the AI system is located in a smart phone or a body-worn or body-carried device carried by the recipient throughout the day or otherwise is in close proximity to the recipient throughout the day. In another embodiment, the system is located remotely, and sound that is captured is periodically uploaded to the AI system and the AI system then evaluates the data. In any event, as the AI system listens in to the recipient's real-world conversations, certain anomalies will be detected by the AI system. One example could be a recipient who has trouble hearing high-frequencies at low levels. This could manifest itself in non-responses to /sh/ or /ss/ sounds spoken at soft levels, and/or misunderstandings of words containing these sounds (e.g., mistaking “singles” for “shingles”). The system could be configured to identify such in at least some embodiments.

Initially, consistent with the teachings detailed above, anomalies are detected and identified, but in at least some instances, are not initially identified or categorized as actionable errors. Over time, however the AI system categorizes certain anomalies as persistent problems or otherwise errors.

Again, in some exemplary scenarios, the AI system provides a report or a summary or the like of these errors or occurrences. In some exemplary embodiments, the AI system provides recommendations as to what could be done with respect to map settings to address these errors. Still further, in an exemplary embodiment, the AI system develops appropriate or utilitarian map adjustments. With respect to the aforementioned scenario, in at least some exemplary embodiments, the AI system would initiate or otherwise develop adjustments to the map or a new map where the threshold levels at high-frequencies are raised relative to that which was the case when the errors occurred.

In an exemplary embodiment, once the change has been applied, assuming or otherwise providing that the change was utilitarian or otherwise addressed the underlying problems causing the errors, the rate of anomalies relating to high-frequency sounds at soft levels would decrease, and such a pattern of error and successful response would be reused by the AI in other similar situations (for that recipient and/or others, thus training the AI system). This can constitute training of the AI system.

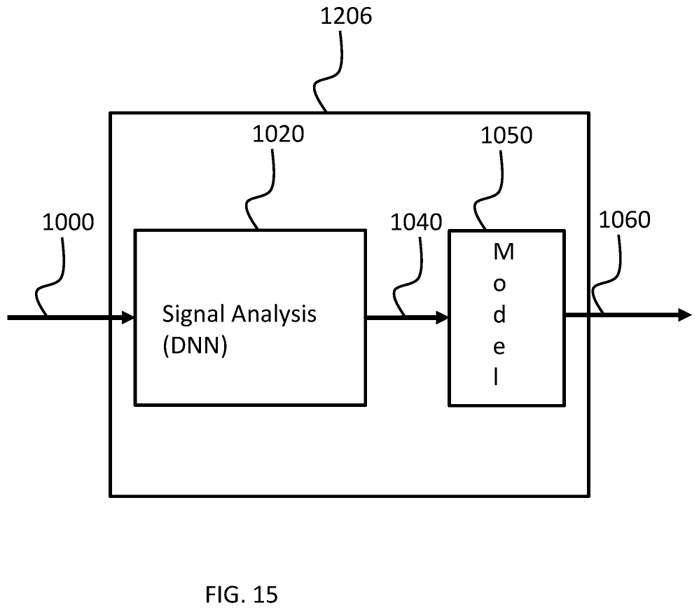

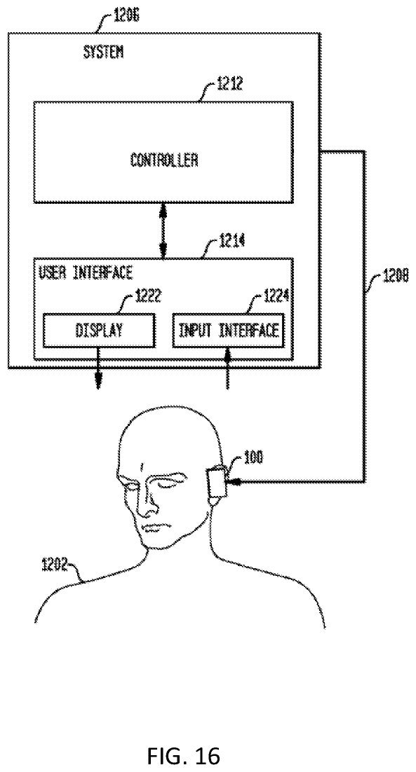

Jumping ahead briefly, FIGS. 15 and 16 provide an exemplary arrangement that can enable and AI system to be trained. Such will be described in greater detail below. However, it is noted that the training of the AI systems detailed herein are applicable both to an individual's system, and an arrangement where a system that is trained for one individual is then used for one or more similarly situated individuals (where those respective systems can further be trained with respect to the individual, in some embodiments).

By way of example only and not by way of limitation, initially, a cycle according to FIG. 4 and/or FIG. 5 can be executed. FIG. 4 has the utilitarian value with respect to the fact that there is some control testing involved, and thus the artificial intelligence system is likely to “learn” faster relative to that of FIG. 5. That said, the two can be used in combination or only one can be used to train the system. With respect to utilization of the two cycles in combination, an initial number of utilitarian cycles can correspond to cycle 4, and then after that, the remaining cycles can correspond to cycle 5. Periodically, one or more cycles according to FIG. 4 can be executed as a sanity check or the like, and so on.

In an exemplary embodiment, an objective right or wrong regime is implemented, and such is utilized to train the artificial intelligence system. Still further, in an exemplary embodiment, a subjective regime can also be implemented and that too can train the artificial intelligence system, separately or in conjunction with the objective regime.

In an exemplary embodiment that utilizes the cycle of FIG. 5 for training purposes, an input that is subjective or objective can be added to the cycle. Indeed, in an exemplary embodiment, the performance monitoring 510 can include facets of at least an objective regime. In any event, after the AI activity is executed, and the maps/settings of the prosthesis are adjusted or otherwise changed, and the recipient engages in use 330, objective tests can be executed to determine whether or not the new settings/maps are better than the old settings/maps. If they are better, then the system can “remember” that these changes were a good thing based on the given input, and then thus use these changes with respect to a given scenario in the future. If they are not better, the system can remember that these changes were not good, and thus might be less likely to utilize these changes at a later date.

Note that different types of input can be fed into the artificial intelligence system beyond mere performance monitoring. By way of example only and not by way of limitation, recipient gender, employment, lifestyle, age, date of onset of deafness, native language, etc., or any other demographic data point that can be statistically useful, can be input into the artificial intelligence system. In embodiments where the trained artificial intelligence system, trained for one or more recipients, for example, is utilized for other recipients, the demographic data can be used by the AI system to determine or otherwise develop changes to a given map or prosthesis settings for that given recipient based on demographic characteristics and/or physiological characteristics associated with that recipient.

In any event, in some embodiments, there is an initial training regime where a statistically significant number of recipients are initially gathered, and the artificial intelligence system is utilized with those recipients. The number of recipients could be 20 or 30 or 40 or 50 or 75 or 100 or 150 or 200 or 250 or 300 or more recipients. Initially, the artificial intelligence system may make changes that are very bad, and based on feedback from the recipient and/or based one objective tests, the artificial intelligence system could learn not to make these changes with respect to a given scenario, at least for a given demographic. In an exemplary embodiment, the aforementioned initial recipients could all be utilized in a controlled or semi controlled test environment, at least initially, so that the initial learning can take place. In an exemplary embodiment, initially, the system could be trained based on objective tests/active tests, and then the system could be further trained by allowing the recipients to interact in controlled or uncontrolled speech environments (a controlled speech environment could be conversations akin to actors reading scripts, where the scripts have words and/or phrases that are known to cause difficulties with respect to people utilizing hearing prostheses; an uncontrolled speech environment could be the utilization of the system during normal lifestyle use—the recipients could be given the partially trained/initially trained system, and then told to go out and conduct their lives as normal, where the system continues its training). In an exemplary embodiment, the training can initially be furthered by presenting the system with a controlled speech environment, and then subsequently and uncontrolled speech environment. Thus, the systems are gradually trained in a manner that reduces the likelihood of a “serious” incorrect decision being made, which might be more likely to occur if the system was initially exposed to the uncontrolled speech environment. That said, embodiments can also include simply exposing the system to an uncontrolled speech environment right off.

Still, embodiments can use the initial controlled and limited number of subjects approach for initial training or even total training for that matter. It is noted that after the system is deemed to be sufficiently trained, such as after the input from the 20 or 50 or 100 or however many initial recipients is achieved and the system trains on those systems, the system may never be trained again or otherwise remains a static system. That said, even after the system is deemed to be sufficiently trained, the training can continue, either in a controlled setting, or in an uncontrolled setting. By way of example only and not by way of limitation, in some exemplary embodiments, the system is applied to recipients in an untrained state, and the given recipients are individually used for training purposes of the system, and over time, the system trains itself to operate according to the teachings detailed herein. Still further by way of example only and not by way of limitation, in some exemplary embodiments, the system is first applied to non-test subject recipients after it has been at least partially trained, or otherwise sufficiently trained, and then each individual recipient trains the system he or she uses, and that training is limited for use with that given recipient. That said, in another exemplary embodiment, the training is not limited for use with a given recipient, but instead, the now extra-trained system, is utilized for other recipients, at least for recipients who are demographically similarly situated to the prior trainer, and so on.

It is to be understood that the concept of training the AI system is not necessarily mutually exclusive with the concept of utilizing the AI system to achieve utilitarian value with respect to improving or otherwise enhancing the recipient's ability to hear. In this regard, any disclosure herein of a method action associated with improving the recipient's ability to hear also corresponds to another disclosure of an exemplary embodiment of performing that action to train the artificial intelligence system, and vice versa.

Alternatively, if the map was not utilitarian or otherwise the changes were not utilitarian or did not address the underlying problem, the rate of anomalies relating to high-frequency sounds at soft levels would not necessarily decrease, and could even potentially be increased, or otherwise might decrease to a statistically insignificant value and such a pattern of error an unsuccessful response would not be reused by the AI in other similar situations.

The pattern described above could be, implemented in an expert rules-based system. The AI comes into its own when more complex, interrelated problems are presented.

In any event, with respect to the demographic data, in an exemplary embodiment, there is the action of identifying recipients who are similarly situated to other recipients who were utilized to train a given system, and utilizing that train system for those similarly situated recipients. Thus, in an exemplary embodiment, there could be two or three or four or five or six or seven or eight or nine or 10 or 11 or 12 or 13 or 14 or 15 or more systems having different training which are respectively used for some recipients and not others. That said, in an exemplary embodiment, a single system can be sufficiently trained that can identify the given demographic, and apply certain features to that demographic at the exclusion of other features. Again, in an exemplary embodiment, the inputs into the system are beyond that associated with speech voice data. Demographic data and the like can be inputted as well.

In an exemplary embodiment, the underlying data utilized by the artificial intelligence system are not linear and/or the results are not linear. Hence the utilitarian value of utilizing an artificial intelligence system.

It is also briefly noted that in at least some exemplary embodiments, the issues associated with a recipient not being able to hear as well as that which otherwise might be the case may not necessarily be parameter based/prosthesis setting based. In an exemplary embodiment, it could be environment and/or another phenomenon. In an exemplary embodiment, problems can arise simply because the recipient has not had his or her cup of coffee in the morning. Problems can arise because the recipient is trying to stop smoking or otherwise going through a midlife crisis. Indeed, consider the scenario of a child, where the child decides that he or she is just going to ignore a certain person, just because. These are not problems associated with the prosthesis or with the settings of the prosthesis. However, the actions associated with such scenarios could be interpreted by a system as being indicative of a problem with hearing. Accordingly, in an exemplary embodiment, the system is “smart enough” to differentiate between environmental problems or non-hearing related problems, and parameter problems.

To be clear, in at least some exemplary embodiments, the teachings detailed herein are limited to the development of setting parameters of a hearing prostheses utilizing the artificial intelligence system. Some exemplary embodiments are specifically limited to the developments or adjustment of maps or otherwise the fitting of a hearing prostheses utilizing the artificial intelligence system. That said, in some embodiments, the artificial intelligence systems are utilized to do other things, such as to also identify possible changes in environment, etc., or used in conjunction with an alternate or a separate artificial intelligence system, that does other things.

Still, it is noted that in at least some exemplary embodiments, the artificial intelligence system can be sufficiently well-trained to distinguish between parameter-based anomalies and non-parameter-based anomalies.

Returning back to hearing problems, an example of a more complex problem could be where the recipient repeatedly has problems discriminating between /e/ and /i/ sounds. The AI would pick up a number of instances where the patient responds inappropriately in a conversation for example if another talker asks, “could you pass the pin?” and the recipient responds with “which pen, the blue one?” The recipient can possibly also ask the speaker to repeat themselves where the sentence contains an /e/ or /i/ sound. As a number of such anomalies are observed by the error detection process, the area is marked as a mistake. Once the mistake is identified, it is then fed into the map development section/map optimizer section, which makes adjustments that have worked for similar issues in the past. The success of this intervention is then monitored by the system, and further changes applied if necessary.

In an exemplary embodiment, artificial intelligence is also utilized to develop the map optimization/map development section. In an exemplary embodiment, lookup tables or the like are utilized. In an exemplary embodiment, there is an algorithm located on computer code that can take the output from the error detection/determination section of the system, and evaluate that output to develop the map.

Early on, these adjustments may be frequent as the map is customized to the recipient and/or as the recipient's brain acclimatizes to the inputs from the implant. Once this period of acclimatization is over or otherwise matures, the map changes are likely to become less frequent.

As seen from the above, embodiments include a fitting system. The system can include an input subsystem, which can be any device or system that can enable the input of data that is used by the system to be inputted into the system. In this regard, in an exemplary embodiment, the input subsystem can be a microphone that is in signal communication to the system either via a wired connection or a wireless connection. In an exemplary embodiment, the input subsection provides captured sound for data based on the captured sound (a signal modified or the like from a microphone) to the system, where the data is recorded/saved and/or analyzed in real time.

The input subsystem can instead be a component that receives a signal from a microphone and need not necessarily include the microphone. In this regard, in an exemplary embodiment, the input subsystem could include a jack or the like that is configured to receive a comparable jack from a microphone. Alternatively, and/or in addition to this, the jack could be a jack that receives input from a memory device or the like or otherwise a device that has stored there on the data. In an exemplary embodiment, this could be a jack that communicates with the output of a tape recorder or an MP3 recording device, etc. In an exemplary embodiment, the input subsystem can receive data from a smart phone or the like such as via a wired or wireless connection. Thus, the input subsystem can be, for example, a Wi-Fi based system that is configured to receive RF frequency transmissions from a remote device, such as the smart phone or the smartwatch, etc. That said, the input subsystem can be or otherwise include the smart phone or smart handheld computer or even a smartwatch in some embodiments. (Indeed, as noted above, the entire system could be operated on a smart phone platform in some embodiments.) Any device or system that can enable the input of data so that the system can perform its functions can be utilized in at least some exemplary embodiments. In an exemplary embodiment, the input subsystem can enable one or more of the method actions detailed herein associated with the capture of sound and/or the capture of speech sound. It is also noted that in at least some exemplary embodiments, the input subsystem can have data logging capabilities of the like. That is, the input subsystem can also be configured to receive input indicative of data that is not based on an audio signal. By way of example only and not by way of limitation, the inputs subsystem can be configured to receive time data, locational data of the recipients, data associated or otherwise indicative of the current settings of the hearing prosthesis (e.g., volume, gain, microphone directionality, noise cancellation, etc.). Indeed, in some embodiments, the input suite can receive data indicative of whether or not the prosthesis is even being used. By way of example only and not by way of limitation, consider a temporal period lasting two or three hours where the recipient does not have his or her hearing prostheses functioning or otherwise where the hearing prosthesis is not functioning. The data associated with the ambient sound could yield, when analyzed, a plethora of errors that are related to the fact that the recipient can hear nothing around him because the hearing prosthesis is not on and thus have nothing to do with the map setting. Thus, the input subsystem can enable one or more of the method actions detailed herein associated with handling the data to be inputted into the system or otherwise used by the system. In at least some exemplary embodiments, the inputs subsystem corresponds to the machine that is utilized to capture the voice while in other embodiments the inputs subsystem can correspond to a device that interfaces with machine captures the voice. Thus, in an exemplary embodiment, the input subsystem can correspond to a device that is configured to electronically communicate with the machine. In some embodiments, the input subsystem can be the microphone and associated components of the device 240 above while in other embodiments, as noted above, the inputs can correspond to sound captured by the hearing prosthesis, and thus can include the sound capture component of the hearing prosthesis and associated components.

Both the microphone of the hearing prosthesis and the microphone of the device 240 can be utilized in conjunction and together as the input subsystem. In an exemplary embodiment, the microphone of the hearing prosthesis can be utilized to capture the sound, and the hearing prosthesis can transmit a radiofrequency signal to device 240 that will be received by the device 240, which signal is based on sound captured by the hearing prosthesis. This can be a streamed audio signal from the hearing prostheses to device 240. The RF communication components of the smart phone, would thus be also included in the input subsystem.

Irrespective of whether or not the prosthesis is utilized as part of the input subsystem, in an exemplary embodiment, the input subsystem (or the input/output subsystem, as will be described in greater detail below) is in signal communication with a hearing prosthesis of the hearing-impaired person.

In an exemplary embodiment of the system, the system also includes a processing subsystem. In an exemplary embodiment, the processing subsystem is a microprocessor-based system and/or can be a computer system based system that can enable one or more of the actions associated with analyzing the captured voice/captured sound to execute the teachings detailed herein. In an exemplary embodiment, the processing subsystem can be configured to identify the weakness in the impaired hearing person's map setting that is identified by using the voice and/or the data as latent variables. In this regard, in an exemplary embodiment, the processing subsystem can be configured to execute any one or more of the analysis and/or determination functions and/or evaluating functions and/or identification functions and/or processing functions and/or classifying functions and/or recommending functions detailed herein. In an exemplary embodiment, the processing subsystem can do this in an automated fashion. In an exemplary embodiment, the processing subsystem is the AI based system detailed herein/functions as such.

In an exemplary embodiment, the system also includes an output subsystem. In an exemplary embodiment, the output subsystem can correspond to the input subsystem while in other embodiments the output subsystem is a separate from the inputs subsystem. In this regard, the output subsystem can correspond to a personal computer, or any of the components associated with the inputs subsystem detailed above. Thus, in an exemplary embodiment, the system can include an input subsystem and an output subsystem and/or an input/output subsystem where, with respect to the latter, input and output subsystems are combined. In an exemplary embodiment, the output subsystem corresponds to the device that provides the output of FIG. 3. In an exemplary embodiment, the output subsystem corresponds to the device that enables the execution of the remapping of the prosthesis. In an exemplary embodiment, the output subsystem can correspond to a device that includes a jack that can be placed into wired communication with the hearing prostheses so as to transfer the map to the prostheses. In an exemplary embodiment, the output subsystem can be a Wi-Fi system. In an exemplary embodiment, the output subsystem can instead be a computer-based system that sends an email or a text message indicating the results of the analysis. In an exemplary embodiment, the output subsystem can be a USB port or the like that enables the message or the report where the new map data to be outputted. In an exemplary embodiment, the output subsystem can be the computer screen of the device 240. The report can be presented on that screen in an exemplary embodiment. In an exemplary embodiment, the new map settings can be displayed on that screen. The output subsystem can also include a speaker or the like.

Any of the output components of a smart phone or a smart watch etc., can be utilized in some embodiments.

Any device, system, and/or method that will enable the output subsystem to output data having utilitarian value with respect to implementing the teachings detailed herein or otherwise that can enable the teachings detailed herein can be utilized in at least some exemplary embodiments.

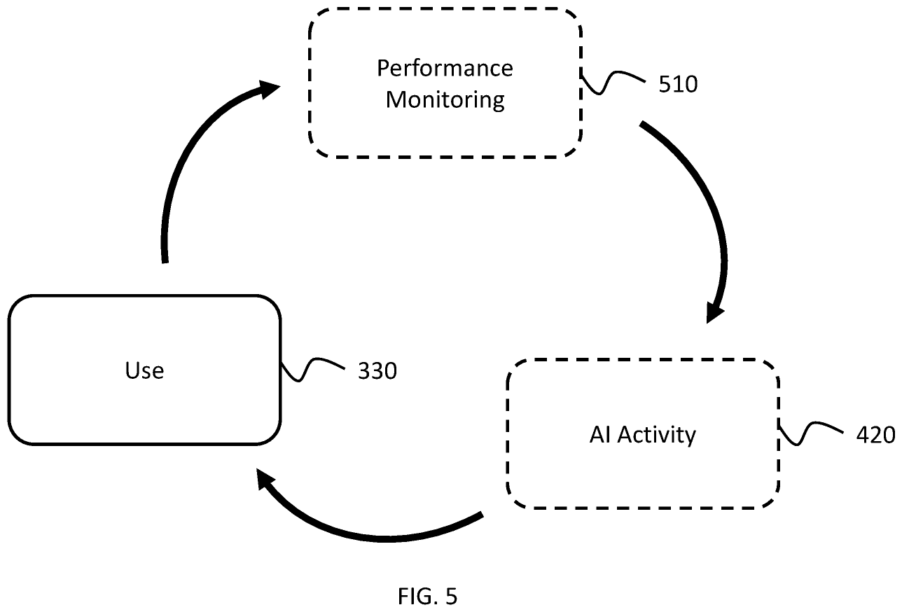

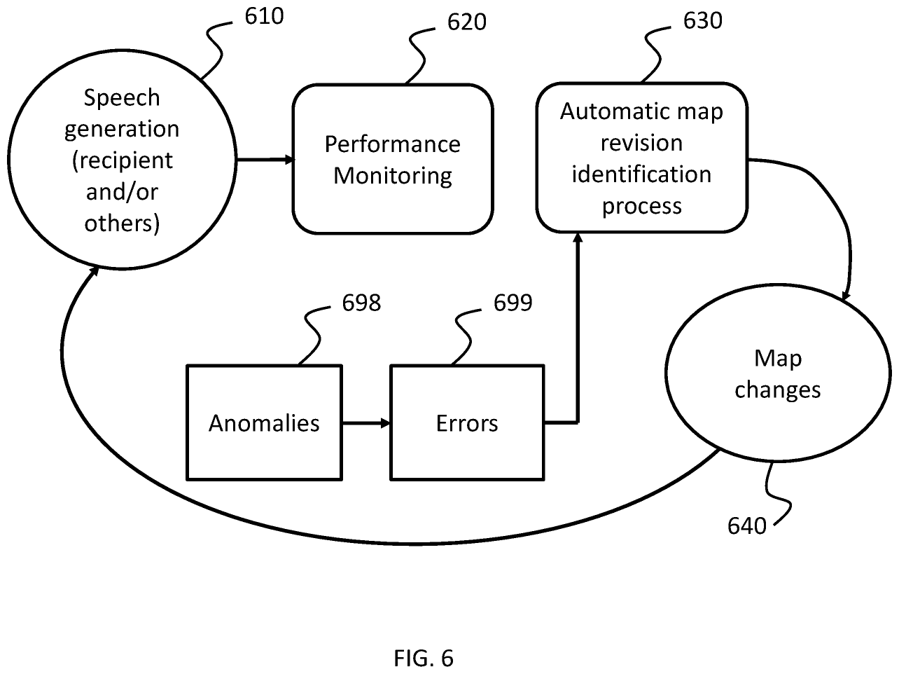

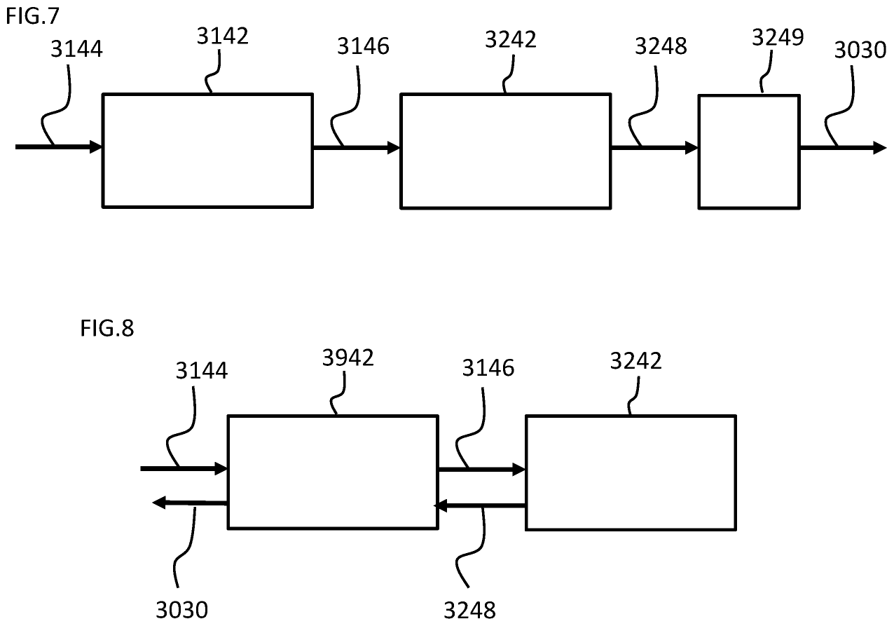

FIG. 7 provides a black-box schematic of an embodiment where the input subsystem 3142 receives input 3144, and provide the input via communication line 3146 (which can be via the internet, or hard-wired communication in the case of the system being on a laptop computer) to processing subsystem 3242, which communicates with the output subsystem 3249 via communication line 3248 (again, internet, hardwired, etc.), where the output is represented by 3030. FIG. 8 provides an alternate embodiment which instead utilizes an input/output subsystem 3942. To be clear, the entirety of the components of FIGS. 7 and/or 8 can reside in a smart phone or a smart watch and/or the hearing prosthesis of FIG. 1 or a variation thereof or another hearing prosthesis (e.g., a middle ear implant, or a bone conduction device). Also, as noted above, a retinal implant could be the basis of these components. Any sensory prosthesis can be the basis for such.

In view of the above, it can be seen that in an exemplary embodiment, there is a fitting system, such as either of the two systems depicted in FIGS. 7 and 8. The fitting system can be for any type of sensory prostheses, such as, for example, a cochlear implant, a retinal implant, etc. The system includes a communications subsystem including at least one of an input subsystem and an output subsystem or an input/output subsystem. The communications subsystem can be that of a smart phone or a personal computer, or a hearing prosthesis, etc. In an exemplary embodiment, the communication subsystem is split between the hearing prosthesis and the smart phone. In this regard, in an exemplary embodiment, the microphone of the hearing prostheses is used as the input subsystem, and the output componentry of the smart phone is utilized as the output subsystem.

In an exemplary embodiment of the fitting system, the system includes a processing subsystem, wherein the processing subsystem is configured to automatically develop fitting data for a hearing prosthesis at least partially based on data inputted via the communications subsystem.

In an exemplary embodiment of the fitting system, the fitting system is configured to develop the fitting data for the hearing prosthesis by analyzing a linguistic environment metric inputted into the communications subsystem. Further, the fitting system can be configured to develop the fitting data for the hearing prosthesis by analyzing a linguistic environment metric inputted into the communications subsystem and a non-listening metric inputted into the communications subsystem or another subsystem (e.g., head turning, lack of head turning, eye movement, etc.—a device can be used to capture such actions or inactions, such as an accelerometer and/or a camera, etc.). In an exemplary embodiment, the former can be a result of the microphone of the hearing prostheses and/or the portable electronics device capturing sound exposed to the recipient. In an exemplary embodiment, the former can be the result of the hearing prostheses wirelessly transferring an audio signal or otherwise data based on sound captured by the microphone of the hearing prosthesis whether processed or otherwise, to the portable handheld electronics device. In an exemplary embodiment, the former can correspond to the downloading or otherwise transferring of a recording of ambient sound captured by any particular machine that can enable such, such as for example, a tape recorder or other recording device, into the communication subsystem. In embodiments utilizing both, the fitting system can use one or both as data upon which is relied to fit the prosthesis.

Thus, in an exemplary embodiment of this exemplary embodiment, the system includes a sub-system including at least one of the hearing prosthesis or a portable body carried electronic device (e.g., smartphone, smartwatch, etc.), wherein the hearing prosthesis is configured to output data indicative of a linguistic environment of the recipient (e.g., via a wired or wireless signal) and the portable electronic device is configured to receive data indicative of the linguistic environment of the recipient, and the linguistic environment metric is based on the at least one outputted data or the received data. Again, in some embodiments, the microphone of the smart phone can be utilized in totality to capture the ambient sound, the microphone of the hearing prostheses can be used to capture the sound in totality, or combination of the two can be used. With respect to the latter, the system can be configured to analyze a given input signal and select the best signal from between the two for analysis by the processing system. For example, the system can evaluate the data captured by the two separate microphones, and select the data that has the best signal to noise ratio for a given segment. For example, seconds 1, 2, 3, 4, 5 of sound can be based on the sound captured by the microphone of the hearing prostheses, seconds 5.1, 5.2, 5.3, 5.4, 5.5 and 5.6 can be based on the microphone of the smart phone, and then seconds 5.7 to 50 can be based on the microphone of the hearing prostheses, and so on. Thus, the system can be configured to evaluate multiple sets of data and pick and choose which data is the best base on fine parsing.

Again, in an exemplary embodiment, the sound can be captured by the hearing prosthesis and streamed real time and/or provided in packets to the portable body carried device, and/or the sound can be captured by the portable body carried device.

Note also that in some embodiments, the subsystem includes the hearing prosthesis and a non-portable body carried electronic device separate from the hearing prostheses. In an exemplary embodiment, the hearing prosthesis can be configured to record or otherwise store the sound captured by the microphone or store data that is based on that sound (e.g., processed data), and then periodically or intermittently or based on another schedule, download or enable the downloading of that store data to a personal computer or to a remote device via the Internet.

Still further, in an exemplary embodiment, where the sub-system includes the portable electronic device, the portable electronic device is a smart device (e.g., smartphone), and the processing subsystem is at least in part located in the smart device. In an exemplary embodiment, the smart device can perform a first level of processing, and another device, a remote device for example, could perform a second level of processing, all of which can be utilized to develop the data detailed herein which is developed by the AI system. That said, in an exemplary embodiment, the AI system is entirely based in the smart device.

Consistent with the teachings detailed above, in an exemplary embodiment, the processing subsystem is an expert sub-system that includes factual domain knowledge and clinical experience of experts as heuristics, and the expert sub-system is configured to automatically develop the fitting data based on the linguistic environment metric.

Embodiments of the expert system are described in greater detail herein. That said, it is also noted that in an exemplary embodiment, the processing subsystem is a neural network, such as, for example, a deep neural network, and the neural network is configured to automatically develop the fitting data based on the metric. As with the expert system, additional features of some embodiments of this will be described in greater detail below.

In an exemplary embodiment where the processing subsystem is an expert sub-system of the system, the subsystem can include code of and/or from a machine learning algorithm to analyze the metric, and the machine learning algorithm is a trained system trained based on a statistically significant population of hearing impaired persons.

Consistent with the teachings detailed above, in some embodiments, the fitting system is a completely autonomous system, and, in some embodiments, the fitting system is configured to automatically develop the fitting data based effectively on or totally on passive error identification. Thus, in an exemplary embodiment, some of the fitting data can be partially based on a phoneme test or an audiogram, but the data can still be effectively based on the passive error identification (note that the audiogram and the phoneme test are not passive error identifications.

Again, consistent with the teachings detailed herein, at least some exemplary embodiments are based entirely on data that is passively collected while the recipient is utilizing the hearing prosthesis to hear. This is not to say that some embodiments cannot use a combination of this passively collected data and other data, such as actively collected data, such as, for example, the results of a test or the like, as well as subjective input and other things, some of which will be described in greater detail below. This is to say that the fitting data is developed at least in part in some embodiments based on passive error identification. As will be detailed below, there can be hybrid fitting systems which analyze both passively acquired data and the results of actively acquired data (e.g., testing), to implement the teachings herein.

Still, in some embodiments, the system is configured to automatically develop fitting data for the hearing prosthesis effectively based solely, and in some embodiments, based solely, on a performance of a recipient of the hearing prosthesis.

As noted above, the actions of collecting the data occur at least in part after the initial device activation session/initial fitting session or otherwise after initial device turned on. Accordingly, in at least some exemplary embodiments, the hearing prosthesis is at least partially fitted to the recipient. In an exemplary embodiment, a map developed at least in part based on subjective and/or objective data associated with the recipient is loaded into the hearing prostheses, which map is utilized to process sounds to evoke a hearing percept at the same time that the sounds are captured to develop the data to be used by the system. In an exemplary embodiment where the fitting system develops a new map or otherwise develop fitting data for the prostheses, this new map/fitting data constitutes a replacement map or an adjustment to an existing map of the hearing prostheses. Thus, in an exemplary embodiment, the system is configured to automatically develop revised fitting data for the hearing prosthesis. Note further that in an exemplary embodiment, as will be detailed below, even after the initial fitting, there can be a subjective content to the activities associated with developing the revised fitting data. Additional details of this will be described below, but it is briefly noted that in an exemplary embodiment, the system is configured to automatically develop revised fitting data for the hearing prosthesis based on subjective preference input from the recipient about the developed fitting data. By way of example only and not by way of limitation, in an exemplary embodiment, the artificial intelligence system could develop fitting data, and this fitting data (revised fitting data) could be used to refit the hearing prostheses, and in the recipient could say that he or she hates a certain aspect thereof, and then the AI system could reevaluate the fitting and revise that revised fitting data for use in the prostheses. Still further by way of example only and not by way of limitation, in an exemplary embodiment, prior to any activities of the artificial intelligence system, the artificial intelligence system could take into account that the recipient is uncomfortable hearing it certain decibel levels with certain decibel levels falling within a range of frequencies, or otherwise just does not want to hear certain frequencies for whatever reason, and thus the system could take this into account when analyzing the passively acquired data.

From the above, it can be seen that the systems and/or the teachings detailed herein can be utilized in conjunction with subjective input. Systems based solely on performance, whether actively determined through tests, or passively determined through an automated error detection process, fit the hearing prosthesis based on features associated entirely with performance rather than preference. Some exemplary embodiments are such that the recipient's subjective preference is taken into account by allowing an input into the map change. By way of example only and not by way of limitation, after an automated map change has been applied, the patient could be asked to rate the update on a 1 to 5 scale. This input is not burdensome and could be made an optional part of the process. In some embodiments, this rating can be utilized to further train the system for that recipient, while in other embodiments, this rating could be utilized across the board for a statistically significant group of people having demographic characteristics relevant to one another. In an exemplary embodiment, the subjective data can be simply used to override any changes that were made. Still further, in an exemplary embodiment, the subjective input can be utilized in the overall analysis, and the subjective input need not necessarily be sought every time an analysis occurs. For example, if the recipient simply does not like to hear certain frequencies, at least not at certain amplitude levels, this subjective fact can be utilized in the processing or evaluation for possibly the entire period of time that the system is utilized well after the system first “learns” of this.

As noted above, the embodiments of FIGS. 7 and 8 can represent a fitting system. Consistent with the teachings detailed above, in some embodiments, there is a system that is not necessarily a fitting system, but instead a system that develops recommendations or otherwise outputs summaries or reports indicative of an analysis associated with the input. Accordingly, in an exemplary embodiment, any disclosure herein of a fitting system or features associated with fitting corresponds to a disclosure of an alternate embodiment where the system is not a fitting system, but instead a hearing improvement analysis system/recommendation system. The system need not necessarily develop fitting data or otherwise be a system that fits a prosthesis, but instead analyzes the input and provides a report or provides information based on the analysis and/or provides a recommendation one changes that should be made or otherwise can be utilitarian if made to a user. Thus, in an exemplary embodiment, any reference herein of an action of fitting a hearing prosthesis or developing fitting data for the hearing prosthesis corresponds to a disclosure wherein an alternate embodiment, there is an action of providing output indicative of the analysis or otherwise providing recommendations based on the analysis. Corollary to this is that any disclosure herein of a method action associated with such corresponds to a disclosure of a device and/or system that is configured to execute such method actions or otherwise has the functionality associated there with.

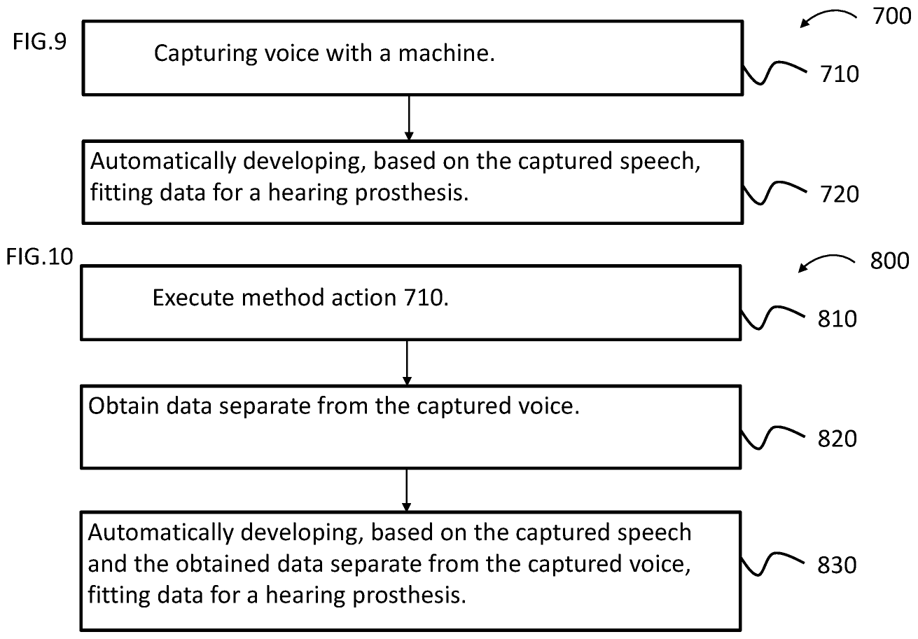

FIG. 9 presents an exemplary algorithm for an exemplary method, method 700, which includes method action 710, which includes capturing voice sound with a machine, such as, for example, implant 100 and/or device 240 detailed above, or the system 210. In an exemplary embodiment, the captured voice can be captured by the microphone of the implant 100. In an exemplary embodiment, the voice can be recorded and stored in the implant 100 and/or in a component associated with the system 210 and/or can be uploaded via element 249 in real time or in partial real time. Any device, system, and/or method that can enable voice capture in a manner that will enable the teachings detailed herein can be utilized in at least some exemplary embodiments. It is noted that in at least some exemplary embodiments, the method further includes analyzing or otherwise reducing the captured voice to data indicative of the captured voice and/or data indicative of one or more properties of the captured voice, which data then can be stored in the implant of the system and/or communicated to a remote server, etc., to implement the teachings detailed herein. The data indicative of one or more properties of the captured voice will be described in greater detail below along with the use thereof. Ultimately, the data obtained in method action 710 can correspond to the linguistic environment measurements/the dynamic communication metrics detailed herein.

Method 700 further includes method action 720, which includes automatically developing, based on the captured speech captured in method action 710, fitting data for a hearing prosthesis.

In an exemplary embodiment, the action of developing of the fitting data is executed by processing the data using a code from a machine learning algorithm. In an exemplary embodiment, the action of developing the fitting data is executed using a neural network. In an exemplary embodiment, the action of developing the fitting data is done using an expert system.

In an exemplary embodiment, the method includes identifying one or more anomalies and/or identifying the identified anomalies as actionable errors using a code from a machine learning algorithm, using neural network or using an expert system, or some form of AI system.

FIG. 10 presents another exemplary algorithm for another exemplary method, method 800, which includes method action 810, which includes executing method action 710. Method 800 also includes method action 820, which includes obtaining data separate from the captured voice. In an exemplary embodiment, the data relates to the use of a hearing prosthesis by a recipient who spoke the captured voice and/or to whom the captured voice was spoken. In at least some exemplary embodiments, the data is logged data that can correspond to the auditory environment measurements, locational data, prosthesis settings or status data, etc. Again, in an exemplary simplest form, the data obtained in method action 820 can be whether or not the hearing prostheses is being used to evoke a hearing percept (e.g., is it on or off). In an exemplary embodiment, method action 820 corresponds to logging data, wherein the logged data is non-voice-based data corresponding to events and/or actions of a recipient of a hearing prosthesis' real world auditory environment, wherein the recipient is a person who spoke the captured voice and/or to whom the captured voice was spoken. Method 800 further includes method action 830, which includes automatically developing, based on the captured speech and the obtained data separate from the captured voice, which data is obtained in method action 820, fitting data for a hearing prosthesis.

Concomitant with the teachings above, in an exemplary embodiment, the machine of method action 710 is a hearing prosthesis attached to a recipient or a smartphone, or a smart watch, or even a microphone associated with the internet of things, or a microphone of a tape recorder, etc. It can be any device that can enable the teachings herein. In an exemplary embodiment, the logged data is indicative of temporal data associated with use of the prosthesis. By way of example only and not by way of limitation, it can be a percentage of a day that the prosthesis is utilized. In an exemplary embodiment, it can be the number of hours per day per week per month etc., that the prosthesis is utilized. In an exemplary embodiment, it is the number of times in a given day or week or month etc. that the prosthesis is turned on and/or turned off or otherwise activated and/or deactivated. In an exemplary embodiment, the data indicative of temporal data associated with use of the prosthesis is associated with the time of day, whether the recipient is awake or asleep, etc. Any temporal data that can be utilized to implement the teachings detailed herein can be utilized in at least some exemplary embodiments.

In an exemplary embodiment, the actions of capturing speech and developing of the fitting data are executed by a system that includes the hearing prosthesis and/or a smart device carried by a recipient of the hearing prosthesis. In an exemplary embodiment, the fitting data that is developed is based entirely on the captured speech. It is noted that this does not mean that all of the fitting data be based on speech, just that the data that is developed be so based.

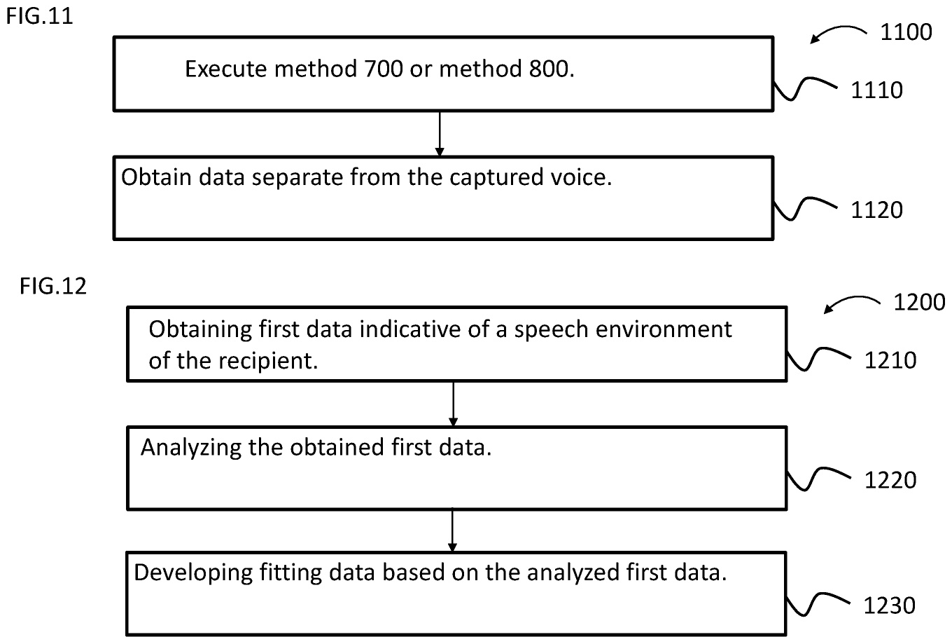

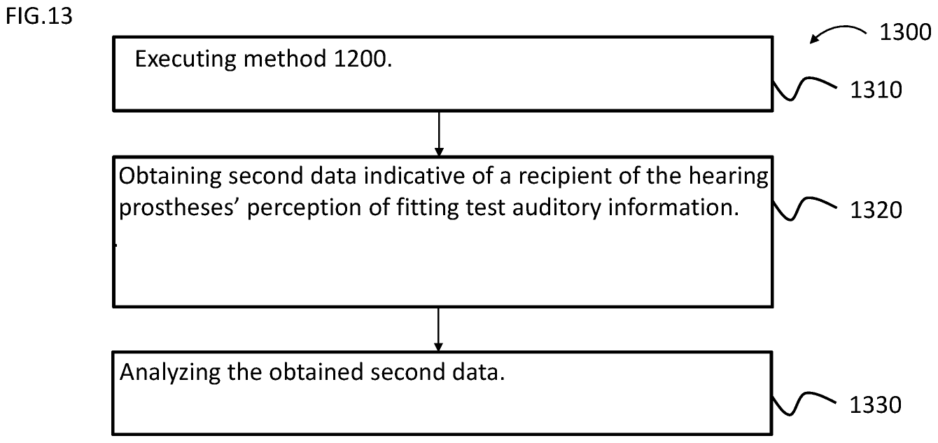

FIG. 11 provides another exemplary algorithm for an exemplary embodiment of an exemplary method, method 1100, which method includes method action 1110, which includes executing either of methods 700 or 800. Method 1100 further includes method action 1120, which includes fitting the hearing prostheses utilizing the fitting data. FIG. 12 provides another exemplary algorithm for an exemplary embodiment of an exemplary method, method 1200, which method includes method action 1210, which includes executing either of methods 700 or 800. Method 1200 further includes method action 1220, which includes automatically adjusting a map of the hearing prosthesis and/or replacing a map of the hearing prosthesis based on the fitting data. It is noted that in this exemplary embodiment of this method, this method does not require all of the fitting data be used.

In an exemplary embodiment of any of the methods detailed herein, the methods can further include automatically determining a recommended change in the recipient's sound environment based on the captured speech and/or sound captured with the captured speech (e.g., background noise/sound can be captured along with the captured speech). By way of example only and not by way of limitation, this can correspond to determining that the recipient should deactivate a noise source, such as a central air-conditioning fan, when speaking to certain members of one's family (or all—in some embodiments, a frequency of the voice of one family member might be too close to the frequency of the fan, which phenomenon does not exist for any of the other family members). In an exemplary embodiment, this can correspond to determining that certain rooms provide better hearing results than other rooms. For example, in an exemplary embodiment, an office worker might be recommended to go to a conference room to have a discussion, as opposed to having a discussion in his or her office or on the open floor, etc. The point is, the features associated with the teachings detailed herein include data collection, which data that is collected can be utilized for the purposes of the developing fitting data, but can also be utilized for other purposes. Thus, the system detailed herein can be utilized to kill two or more birds with one stone. In an exemplary embodiment, it is the artificial intelligence system that executes the action of automatically determining a recommended change in the recipient's sound environment, while in other exemplary embodiments, a system separate from the artificial intelligence system, which system may not be an artificial intelligence system, is utilized to execute this method action.

In an exemplary embodiment, the action of capturing speech is executed during normal, everyday interactions between the recipient of the hearing prosthesis and others. This as opposed to the action of capturing speech that is executed during non-normal non-everyday interactions, such as when the recipient is interfacing with his or her audiologist or the like are working with his or her audiologist to evaluate or improve hearing with the hearing prosthesis, and/or such as when the recipient is executing a self test or a test administered, or under the guidance, or prompted by a caregiver, etc.

An exemplary embodiment of normal everyday interactions could be that which corresponds to a child recipient attending school, an office worker working in an office, a laborer working at a labor site, a machinist working at a machine shop, a restaurant worker working at the restaurant, a person engaging in recreational activities, a person engaging in life-sustaining activities (e.g., shopping, going to the doctors, exercising, etc.).

To be clear, in an exemplary embodiment, normal everyday interactions specifically exclude actions that are exclusively for the purpose of evaluating the recipient's ability to hear or otherwise improving or modifying or changing the hearing prostheses or otherwise developing data therefore are associated there with. A hearing test is not normal everyday interaction.

In an exemplary embodiment, the action of capturing speech is executed in a random manner. In an exemplary embodiment, the speech that is captured and used to execute the teachings detailed herein is random. In an exemplary embodiment, the speech that is captured and used is speech that is not based on a reading. In an exemplary embodiment, the speech that is captured and used is not speech that is speech that is repeated based on what the recipient heard. In an exemplary embodiment, the speech that is captured and used is speech that corresponds to that which would be associated with someone that does not have a hearing prosthesis/is speech that corresponds to that which would be spoken to someone without a hearing prosthesis.

In an exemplary embodiment, the fitting data is based partially on the captured speech and partially based on non-speech data.

In an exemplary embodiment, the fitting data is based partially on sound that is captured with the captured speech. In an exemplary embodiment, the fitting data is partially based on the data that is logged as noted above. In an exemplary embodiment, the fitting data is partially based on locational data. In an exemplary embodiment, the fitting data is partially based on a status or a feature of the hearing prostheses (e.g., a gain setting, whether noise cancellation was activated, etc.).

In an exemplary embodiment, there is any of methods 700 or 800, further comprising the action of at least one of refitting or adjusting an existing fitting map of a hearing prosthesis using the fitting data. In an exemplary embodiment, the refitting or the adjusting of the existing fitting map is based entirely on fitting data that is entirely developed based on the captured speech. Conversely, in an exemplary embodiment, the refitting or the adjusting of the existing fitting map is based entirely on fitting data that is not entirely developed based on the captured speech.

In an exemplary embodiment of any of the methods detailed herein, the action of developing the fitting data is executed with less than A hours of audiogram related testing, phoneme discrimination testing and/or word testing, where A equals 0.1, 0.2, 0.3, 0.4, 0.5, 0.6, 0.7, 0.8, 0.9, 1, 1.25, 1.5, 2, 2.5, 3, 4, 5, 6, 7, 8, 9, 10, 11, 12, 13, 14, 15, 16, 17, 18, 19, 20, 21, 22, 23, 24, 25, 26, 27, 28, 29, or 30 or more. In an exemplary embodiment of any of the methods detailed herein, the action of developing the fitting data is executed within a range of any of the values of A hours of audiogram related testing, phoneme discrimination testing and/or word testing. Accordingly, in an exemplary embodiment, the teachings detailed herein can effectively reduce and/or significantly reduce the testing associated with fitting a hearing prosthesis and/or refitting a hearing prosthesis or otherwise optimizing a map setting of a hearing prosthesis, all other things being equal.

Indeed, in some embodiments, the teachings detailed herein are executed without any of one or more or all of the testings detailed herein.

In an exemplary embodiment of method 700 and/or method 800, the action of developing the fitting data is executed, with respect to testing, with effectively only loudness scaling testing, if any effective testing. That said, in some embodiments, there are other testings that are included. Also, sometimes, there is effectively no loudness scaling testing.

Still, it can be seen that some embodiments include the implementation of a hybrid system that constitutes something in between a fully passive system of error detection. In an exemplary embodiment, the recipient could be involved with or otherwise be given or otherwise participate in some traditional performance tests combined with the passive monitoring of errors according to the teachings detailed herein. By way of example only and not by way of limitation, this could be system initiated or audiologist initiated or other healthcare professional initiated, where with respect to the latter, the results are inputted into the system, there could be one or more of the following tests, audiogram testing and/or development for purposes of detection of the like, phoneme discrimination tests for purposes of discrimination testing, loudness scaling tests for purposes of loudness perception, and/or word tests for purposes of speech perception. Passive monitoring of the type described above in the error detection process could replace some of these tests (audiogram, phoneme discrimination and word testing for example), and the recipient could be asked to run loudness scaling test. The AI system could then take the inputs from both active tests and the passive error detection process as inputs to determine map updates. In an exemplary embodiment, this could have utilitarian value with respect to speeding up the testing process and otherwise resulting in a fitting process that is less burdensome for the recipient. This can also provide a utilitarian interim step before a fully passive system is viable.

That said, embodiments include fitting the hearing prostheses without executing one or more or all of the aforementioned tests. Embodiments can also include fitting the hearing prosthesis utilizing the artificial intelligence systems detailed herein without executing one or more or all of the aforementioned tests.

FIG. 12 presents an exemplary algorithm for another exemplary method, method 1200, which includes method action 1210, which includes obtaining first data indicative of a speech environment of the recipient. Method 1200 further includes method action 1220, which includes analyzing the obtained first data, and includes method action 1230, which includes developing fitting data based on the analyzed first data.

Briefly, it is noted that any disclosure of any method action herein or any functionality of a device and/or system corresponds to a disclosure of a non-transitory computer-readable medium having recorded thereon, a computer program for executing that method action were that functionality, etc. Accordingly, an exemplary embodiment includes a non-transitory computer-readable media having recorded thereon, a computer program for executing at least a portion of a hearing-prosthesis fitting method, the computer program including code for obtaining first data indicative of a speech environment of the recipient (and/or code for enabling a obtaining of first data indicative of speech environment, which could be code that enables the dispositioning or otherwise placement of a received audio signal or a received data set that contains audio recordings, etc., within a computer system), code for analyzing the obtained first data and code for developing fitting data based on the analyzed first data.

In an exemplary embodiment of method 1200 and thus the code associated there with, the action of obtaining first data indicative of a speech environment recipient can include capturing sound of the ambient environment of the recipient (or code enabling such). This can also correspond to the action of receiving a recording or the like of sound of the ambient environment which was obtained during a prior temporal period than the temporal period associated with method action 1210. Method action 1200 can be executed according to any of the teachings detailed herein. This is also the case with respect to method action 1220, where method action 1220 can be executed utilizing the artificial intelligence system detailed herein or variations thereof. Method action 1230 can likewise be executed according to any of the teachings detailed herein, and can be executed utilizing the artificial intelligence system, or can be executed based on the outputs of the artificial intelligence system. Indeed, in an exemplary embodiment, there is an intervening action in method 1200, which includes, after method action 1220, outputting the analysis. In an exemplary embodiment, a clinician or the like can evaluate the output of the analysis, and then utilizing the outputs of the analysis, execute method action 1230. Still, in an exemplary embodiment, method actions 1220 and 1230 are performed automatically by and AI system.

In an exemplary embodiment of the computer readable medium associated with method 1200, or any of the other methods actions detailed herein, the media is for a self-fitting method for the hearing prosthesis that enables a recipient thereof to self-fit the hearing prosthesis. Accordingly, in an exemplary embodiment of method 1200, method 1200 is a method of self-fitting the hearing prosthesis, where the recipient self-fits the hearing prosthesis by executing method 1200 or at least utilizing a device that enables method action 1200. This as contrasted to clinician software that is utilized by a clinician to fit a hearing prosthesis based on inputs. In an exemplary device that is utilized by a clinician, data is obtained regarding features associated with the recipient, such as threshold and comfort levels, and other physiological features of the recipient, and the software can develop a map or the like utilizing, for example, a genetic algorithm, which map will be outputted to the hearing prosthesis, and thus fitting the hearing prosthesis in an automated manner under the guise or otherwise under the control or with the assistance of the clinician/audiologist. Conversely, method 1200 can be executed without any input whatsoever by a clinician/audiologist. Indeed, in an exemplary embodiment, method action 1200 or any of the other method actions detailed herein for that matter are executed without a clinician/audiologist being involved.

To be clear, at least some exemplary embodiments of the teachings detailed herein corresponds to autonomous fitting. In an exemplary embodiment, the systems and devices disclosed herein are autonomous fitting systems. In an exemplary embodiment, the method actions, at least some of them, disclosed herein are autonomous fitting methods. The devices and/or systems disclosed herein can correspond to interventionless fitting systems/the methods disclosed herein can correspond to interventionless fitting methods. At least some exemplary embodiments enable fitting of the prosthesis (or refitting—any disclosure herein of fitting corresponds to a disclosure of refitting, and vice versa, unless otherwise noted) without an audiologist or otherwise without intervention by a healthcare professional. Indeed, in an exemplary embodiment, there is a hearing prosthesis that has never been fitted by an audiologist or which is fitted to a recipient, where the prosthesis has never been adjusted by an audiologist (with respect to a given recipient—generic adjustments can be made for a general populace). Still further, as detailed herein, there are hearing prostheses in some embodiments, that, after the first activation, have never been adjusted by an audiologist or other healthcare professional with respect to an adjustment made for a specific recipient.

Again, it can be seen that in at least some exemplary embodiments, the teachings detailed herein can utilize in some embodiments, purely, 100%, passive data collection and/or analysis to develop fitting data.

In an exemplary embodiment, the code for analyzing the obtained first data and for developing fitting data is located in a smart portable device.

In an exemplary embodiment, consistent with the teachings detailed herein that utilize artificial intelligence or the like, the media is for an automatic fitting method that enables the automatic fitting of the hearing prosthesis based on the speech environment. Indeed, in an exemplary embodiment, the code for analyzing the obtained first data is code of or from a trained machine learning algorithm, some additional details of which will be described below.

In an exemplary embodiment, there is an exemplary method, method 1300, that includes method action 1310, which includes executing method 1200. Method 1300 also includes method action 1320, which includes obtaining second data indicative of a recipient of the hearing prosthesis' perception of fitting test auditory information. In this regard, as noted above, in an exemplary embodiment, the passive data can be utilized in conjunction with active data collection techniques, such as that resulting from testing, to develop a map or otherwise revise a map for the prosthesis.

Method 1300 also includes method action 1330, which includes analyzing the obtained second data. In an exemplary embodiment, this can be executed by the artificial intelligence system and/or can be executed by a clinician or an audiologist. In an exemplary embodiment of the former, the obtained second data can be inputted into the artificial intelligence system so that the artificial intelligence system can evaluate that data along with the first data to develop a map or otherwise provide recommendations, or give a summary, etc. In an exemplary embodiment of the latter, the clinician analyzes the obtained second data, and then provides the analysis to the artificial intelligence system, which can evaluate the first data along with the results of the analysis from the clinician. Indeed, in an exemplary embodiment, both scenario can take place. The artificial intelligence system can evaluate the second data or otherwise analyze the second data in conjunction with the clinician or the like analyzing the second data as well, and the results of both analyses can be utilized by the artificial intelligence system and/or by the clinician to execute the developments of the map and/or develop the recommendations with a summary, etc. It is further noted that in an exemplary embodiment, the artificial intelligence system could analyze the first data, and then develop the map or otherwise provide the recommendations or summary, and then the clinician/audiologist could analyze the second data and make changes to the output from the artificial intelligence system, whether that be making modifications to the map developed by the artificial intelligence system or revising or extending or changing or even the leading the recommendations or summary from the artificial intelligence system.

Consistent with method 1300, in an exemplary embodiment, there is thus a computer readable medium that includes code for operating a system executing at least a part of method 1200 in a fitting test mode, code for obtaining second data indicative of a recipient of the hearing prosthesis' perception of fitting test auditory information obtained while operating in the fitting test mode, and code for analyzing the obtained second data. In an exemplary embodiment, the code for developing the fitting data is also code for developing such based on the analyzed second data. Conversely, in an exemplary embodiment, separate codes are utilized.

Note also, in an exemplary embodiment, there is not necessarily code for operating the system in a fitting test mode. Instead, in an exemplary embodiment, the analyzed results of the separate fitting test can be inputted into the system, as detailed above.

In an exemplary embodiment, the code for obtaining second data enables a system executing at least part of the method to obtain the second data via active activity on the part of the recipient of the hearing prosthesis. In an exemplary embodiment, the code can enable an interactive system to prompt or otherwise receive input indicative of a recipient repeating words, etc., and the system can analyze what the recipient says.

In an exemplary embodiment, the code for the enabling of the obtaining of the second data enables a system executing at least part of the method to obtain the second data in an interactive manner with the recipient of the hearing prosthesis.

In this regard, in an exemplary embodiment, the system can include a speaker that outputs high quality audio or even less than high quality audio, corresponding to speech, which has words, and the recipient can be prompted to repeat the words that he or she hears, and the system can capture the words utilizing a microphone or other sound capture system, and then evaluate the captured sound to identify possible hearing problems or otherwise identify errors associated with the feedback from the recipient. Alternatively, in an exemplary embodiment, the system can receive nonverbal input. In an exemplary embodiment, the recipient can touch a touchscreen indicative of a word that the recipient believes he or she heard. Any regime that can enable an interactive exchange with a recipient can be utilized in at least some exemplary embodiments.

Note also that in an exemplary embodiment, the system need not necessarily have an output component. In an exemplary embodiment, input indicative of the underlying “questions” of a hearing test is inputted into the system (e.g., the code 1032042 for hearing test 103042 is inputted, and the system recognizes that the hearing test include certain phrases and words, etc.), and then input indicative of the recipient responses is inputted.

Again, consistent with the teachings detailed herein, in an exemplary embodiment, the code for analyzing the data can be based on artificial intelligence (again, described in greater detail elsewhere).

In an exemplary embodiment, the code for analyzing the obtained second data is located in a smart portable device

As noted above, in an exemplary embodiment, the teachings detailed herein can be utilized to implement fitting of a hearing prostheses based on relatively limited amounts of testing if any testing at all. As noted above, the testing is quantified in terms of temporal benchmarks. Conversely, at least some exemplary embodiments enable the fitting of a prostheses based on relatively massive, temporally speaking, amounts of data. By way of example only and not by way of limitation, in an exemplary embodiment, there is a method that comprises fitting a hearing prosthesis or a vision prosthesis or any particular type of sensory prosthesis, based on at least B number of hours of sensory prostheses recipient participation obtained within a B×X hour period or a C period. In an exemplary embodiment, B is 50, 75, 100, 125, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2250, 2500, 2750, 3000, 3500, 4000, 4500, 5000, 5500, 6000, 7000, 8000, 9000, or 10000 or more or any value or range of values therebetween in 1 hour increments (777, 2001, 104 to 2222 hours, etc.). In an exemplary embodiment, X is 5.0, 7.5, 10.0, 12.5, 15.0, 20.0, 25.0, 30.0, 35.0, 40.0, 45.0, 50.0, 55.0, 60.0, 65.0, 70.0, 75.0, 80.0, 85.0, 90.0, 95.0, 100.0, 110.0, 120.0, 130.0, 140.0, 150.0, 160.0, 170.0, 180.0, 190.0, 200.0, 225.0, 250.0, 275.0, 300.0, 350.0, 400.0, 450.0, 500.0, 550.0, 600.0, 700.0, 800.0, 900.0 or 1000.0 or more or any value or range of values therebetween in 0.1 increments. In an exemplary embodiment, C is 50, 75, 100, 125, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2250, 2500, 2750, 3000, 3500, 4000, 4500, 5000, 5500, 6000, 7000, 8000, 9000 or 10,000, 11000, 12000, 13000, 14000, 15000, 16000, 17000, 18000, 19000, 20,000, 21,000, 22,000, 23,000, 24,000, 25,000, 30,000, 40,000, 50,000, 60,000, 70,000, 80,000, 90,000, or 100,000 or more, or any value or range of values therebetween in 1 hour increments. In an exemplary embodiment, the period begins at the time that the hearing prostheses is first activated and utilized to evoke a hearing percept in the recipient. In an exemplary embodiment, the period begins at the time that the hearing prosthesis recipient participation begins to be obtained.

In an exemplary embodiment, for example, the period begins with device activation on day 30 (30 days after implantation of a cochlear implant/30 days after the closure of the surgical operation to implant the cochlear implant). The recipient goes off and utilizes the device for two or three or four or five or six or seven or eight or nine or 10 or 15 or 20 or 30 or 40 or 50 or 60 days or more, where the recipient initially acclimates himself or herself to the device. Then, participation commences, by executing the teachings detailed herein to record and analyze data. It is noted that it is possible that recording of ambient sound could occur in the aforementioned period while the recipient acclimates himself or herself. If this recording is not utilized for the evaluations detailed herein, it does not constitute recipient participation. Recipient participation begins when data is collected that is used. If the data is collected and not used, that does not constitute recipient participation.

Thus, in the aforementioned example, the recordings obtained at day 57 are utilized in the teachings detailed herein (which analysis could first occur on day 60 utilizing a regime that has a three-day upload. Alternatively, utilizing the system that analyzes the sound in real time, an analysis can begin on day 57. In any event, day 57 begins the period of recipient participation. From that date, irrespective of how much of recording or real-time sound capture is utilized to the analysis, the larger period begins. Thus, at day 422 (1 year after day 57), 8,760 hours will have elapsed within that period, and if the prosthesis is fitted on that day, or otherwise fitted later, but the data utilized to fit the prosthesis goes no further than day 422, and if a total number of 700 hours or more of that time constitutes hearing prosthesis recipient participation (e.g., 700 hours or more of recording time, however intermittently, was utilized to develop the fitting), the fitting would be based on at least 700 hours of hearing prostheses recipient participation obtained within an 8760 hour period. Note that it is possible that the prosthesis could have been fitted or refitted one or two or three or four or five or six or seven or eight or nine or 10 or 15 or 20 or 30 or 40 or 50 or 60 or 70 or 80 times or any number in one integer increments there between or any range of numbers there between, during the given period (e.g., here, 8,760 hours). Because the fitting utilizes data that is cumulative, the fitting is based on all of that participation. Thus, in an exemplary embodiment where the prosthesis was previously fitted at the 5000-hour mark based on 500 hours of recipient participation, and occurrence will have existed where the prostheses was so fitted and then also fitted using 200 additional hours of recipient participation, and thus fitted based on at least 700 hours of recipient participation.

In at least some exemplary embodiments, the fitting of the hearing prosthesis is executed based on at least 700, 800, 900 or 1000 hours or more of hearing prosthesis participation obtained within a 4,500 period.

In an exemplary embodiment, at least B number of hours of hearing prosthesis recipient participation occurs without interaction with an audiologist. In an exemplary embodiment, at least B number of hours of hearing prosthesis recipient participation occurs without interaction with a healthcare professional having expertise associated with a hearing prosthesis and/or hearing. In an exemplary embodiment, at least 200, 250, 300, 350, 400, 450, 500, 550, 600, 650 700, 750, 800, 850, 900, 950 or 1000 hours of hearing prostheses recipient participation occurs without interaction of the audiologist and/or the aforementioned healthcare professional.

In an exemplary embodiment, during the B×X or C period, there is no more than D hours of audiologist and/or aforementioned healthcare professional interaction with the recipient, where D is 1, 1.5, 2, 2.5, 3, 3.5, 4, 4.5, 5, 5.5, 6, 6.5, 7, 7.5, 8, 8.5, 9, 9.5, 10, 12.5, 15.0, 17.5, 20.0, 25.0, 30.0, 35.0, 40.0, 45.0, 50.0, 55.0, 60.0, 65.0, 70.0, 75.0, 80.0, 85.0, 90.0, 95.0, 100.0, 110.0, 120.0, 130.0, 140.0, 150.0, 160.0, 170.0, 180.0, 190.0 or 200 or any value or range of values therebetween in 0.1 increments. Accordingly, in an exemplary embodiment, during an exemplary 9000-hour period where there is at least 4 or 5 or 6 or 7 or 8 or 9 hundred hours of recipient participation, there is no more than 2 or 3 or 4 or 5 or 6 or 7 or 8 hours of audiologist interaction and/or aforementioned healthcare professional interaction with the recipient.

Concomitant with the teachings detailed above, in an exemplary embodiment, all of the participation is made up of speech conversation interaction between the recipient and others. This is not to say that other data cannot be utilized, or other recipient actions cannot be utilized. This is to say that during that time period, there is at least for example, 750 hours of participation that is made up of speech conversation interaction between the recipient and others irrespective if there is, for example, 20 or 30 or 40 or 50 hours of participation of some other type.

In an exemplary embodiment, at least Y percent of the hours of participation is made up of speech conversation interaction between the recipient and others, where Y is equal to 5, 5.5, 6, 6.5, 7, 7.5, 8, 8.5, 9, 9.5, 10, 12.5, 15.0, 17.5, 20.0, 25.0, 30.0, 35.0, 40.0, 45.0, 50.0, 55.0, 60.0, 65.0, 70.0, 75.0, 80.0, 85.0, 90.0, 95.0, 100 or any value or range of values therebetween in 0.1% increments.

It is noted that in some exemplary embodiments, the aforementioned temporal periods of speech conversation with others excludes that associated with any healthcare professional, such as an audiologist and/or a healthcare professional having expertise in the field of hearing and/or hearing prostheses.

In an exemplary embodiment, the prosthesis is a cochlear implant and the larger period beings C hours after the last medical procedure associated with full and stable implantation of the prosthesis.

It is noted that the variables are used for convenience and textual economy and that a duplicate variable need not be the same. For example, in the above example, where the larger period is C hours and it begins C hours after the last medical procedure, the first C can be 9000 and the second C can be 500. Of course, the first and second C can be equal.

Accordingly, in an exemplary embodiment, there can be a scenario where the first year or two or more of recipient use of the prosthesis occurs in a traditional manner, with audiologist interaction, etc., and then the subsequent years involve the utilization of these teachings herein. Indeed, in an exemplary embodiment, the teachings detailed herein can be first implemented years or decades after a recipient has first begun utilizing a hearing prosthesis.

An exemplary embodiment includes a device, comprising a processor and a memory. In an exemplary embodiment, this device embodied in a smart phone or a smart watch or a personal computer, or mainframe computer. In an exemplary embodiment, this device is configured to receive input indicative of speech sound. Again, in an exemplary embodiment, this could be via a component that includes a microphone or otherwise is a microphone, or a USB port, or any other communications system that can enable receipt of data. In an exemplary embodiment, the device in general, and the processor in particular, is configured to analyze the input indicative of speech sound and identify anomalies in the speech sound based on the analysis of the input, which anomalies are statistically related to hearing prosthesis fitting imperfections.

In this regard, there exists the hypothetically perfectly fitted hearing prosthesis. This is a hearing prosthesis that is optimized with respect to a map and/or settings to a given recipient. Map features or settings that do not correspond to such or otherwise do not result in a perfectly fitted hearing prosthesis corresponds to fitting imperfections.

The feature of anomalies that are statistically related to hearing prosthesis fitting imperfections corresponds to the ability of the device to differentiate between anomalies that are unrelated to hearing prosthesis fitting imperfections from those that are. In this regard, in accordance with the teachings detailed herein, there is an artificial intelligence system that is configured to learn. The learning is based on trial and error in at least some exemplary embodiments, and thus when the device implements the teachings detailed herein, in at least some exemplary embodiments, it is relying upon statistical analysis. Referring to the teachings detailed above, where an anomaly may be encountered a number of times before it is indicated or otherwise determined to be an actionable error, in an exemplary embodiment, if the anomaly occurs only in a certain scenario, and does not occur in other scenarios, the anomaly may or may not be indicated or otherwise identified as being an actionable error. By way of example only and not by way of limitation, if an anomaly exists only infrequently, and always before a recipient has his or her first cup of coffee, the anomaly might not be identified as an actionable error based on the statistical fact.

In an exemplary embodiment, the device includes code from a machine learning algorithm, a neural network and/or an expert system or some form of AI system to execute the action of analyzing the input and/or identifying the anomalies/identifying the anomalies as actionable errors.

In an exemplary embodiment, the method includes identifying one or more anomalies and/or identifying the identified anomalies as actionable errors using a code from a machine learning algorithm, using neural network or using an expert system, or some form of AI system.

In an exemplary embodiment, the device is configured to analyze the identified anomalies and differentiate between anomalies that are indicative of a hearing problem from those that are not indicative of a hearing problem. Again, in an exemplary embodiment, there can be the occurrence where the recipient does not respond to a question. This can be on purpose, or it could be indicative of a hearing problem. The system utilizing, for example, the artificial intelligence system, which code thereof or the system itself, etc., can be included in the device, and thus could reside on the processor or the like, could differentiate between the two.

In an exemplary embodiment, the device is further configured to analyze the identified anomalies and vet the anomalies for utility to fitting the hearing prosthesis. In this regard, this is somewhat analogous to the aforementioned differentiation between anomalies that are indicative of a hearing problem. Here, there could be errors, and the errors can be indicative of a hearing problem, but it is entirely possible that there is no utility with adjusting the hearing prosthesis in this regard. By way of example only and not by way of limitation, it could be that the recipient only has a single-sided hearing prosthesis and is deaf in both ears (100%). The anomalies could be addressed by adjusting a balance or the like between a bilateral hearing prosthesis, but because the prosthesis is only unilateral, such would be a waste of time, and thus the anomaly is an anomaly that cannot be addressed. Still further by way of example, there could be frequencies that the recipient simply cannot hear, even with a cochlear implant (auditory nerve damage at those frequencies). Thus, adjusting a threshold and/or a comfort level for that frequency would be a waste of time. That said, in some alternate embodiments, the captured frequencies can be moved to different channels of the cochlear implant, which channels are mapped to portions of the cochlea where the recipient still can hear. Thus, the perceived frequencies might be drastically off that which occurs in real life, but a hearing percept can still exist albeit for different frequencies.

In an exemplary embodiment of the aforementioned device, the device can be configured to develop fitting data for the hearing prosthesis based on the vetted anomalies that have utility to fitting the hearing prosthesis.

In an exemplary embodiment, the device is configured to identify the occurrence of repeated errors with respect to discrimination between specific phonemes as part of the analysis of the input and identify such as anomalies. This as contrasted to a device that merely identifies the occurrence of errors with respect to discrimination between specific phonemes. In this regard, as noted above, not only does an exemplary embodiment of a device according to the teachings detailed herein identify an error with respect to a phoneme, it also categorizes and catalogs such and determines that something occurs on a repeated basis/statistically significant basis, which determination can be used in the ultimate determination as to whether or not to categorize such as an actionable error. Consistent with the teachings detailed herein, the device is configured to develop fitting data for the hearing prosthesis based on the identified repeated errors.

In an exemplary embodiment, the device is configured to develop the fitting data based on data comprising fitting settings for hearing prostheses that have alleviated the errors for a statistically significant pool of people. Again, consistent with the teachings detailed herein, the artificial intelligence system is a trained system, and the results of utilization of the system that were successful with respect to one recipient could be utilized for other recipients, at least with respect to recipients who are similarly situated or otherwise demographically similarly situated, etc. More on this below.

In at least some exemplary embodiments, the device is configured to automatically fit and/or refit a hearing prosthesis based solely on the identified anomalies. This is not mutually exclusive with a device that also can fit and/or refit the hearing prosthesis based on other inputs. This device however can do it solely on the identified anomalies. In some exemplary embodiments, the device enables performance-based fitting of a hearing prosthesis. This as differentiated from, for example, test-based fitting of a hearing prostheses.

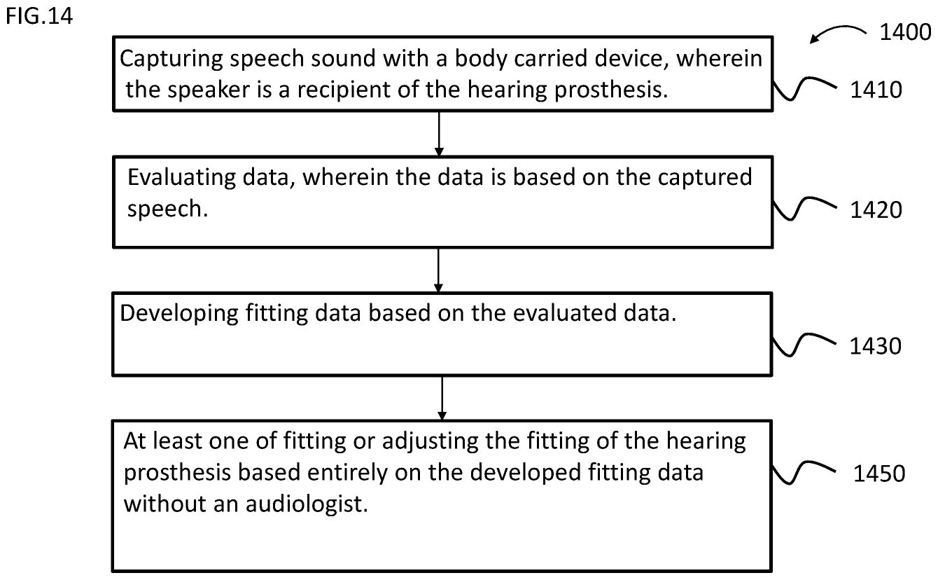

FIG. 14 presents an exemplary algorithm for an exemplary method, method 1400, which includes method action 1410, which includes capturing speech sound with a body carried device, wherein the speaker is a recipient of the hearing prosthesis. The body carried device can be any of the devices detailed herein that can enable such, such as a personal tape recorder, a smart phone, a non-smart phone for that matter and/or the hearing prosthesis itself. Method 1400 further includes method action 1420, which includes evaluating the data, wherein the data is based on the captured speech. This evaluation can be done manually and/or utilizing the system detailed herein. Method action 1400 also includes method action 1430, which includes developing fitting data based on the evaluated data. This can be done manually or utilizing the systems detailed herein. Method action 1400 also includes method action 1440, which includes at least one of at least partially fitting or at least partially adjusting a fitting of the hearing prosthesis based entirely on the developed fitting data without an audiologist. In some embodiments, the fitting is total fitting and/or the adjusting is total adjustments of the fitting.

An exemplary embodiment is based on method 700 or 800. In an exemplary embodiment, a collective action of capturing speech using a machine and automatically developing, based on the captured speech, fitting data for hearing prostheses, is executed in sequence N number of times or at least N number of times. In an exemplary embodiment, N minus Z of the collective actions or at least N minus Z of the collective actions or no more than N minus Z of the collective actions are executed without testing or other affirmative actions on the part of the recipient (other than activating any device or system that is utilized to implement the collective actions). In an exemplary embodiment, Z of the collective actions are executed with testing or other affirmative actions on the part of the recipient. In an exemplary embodiment, N can equal 1, 2, 3, 4, 5, 6, 7, 8, 9, 10, 11, 12, 13, 14, 15, 16, 17, 19, 20, 21, 22, 23, 24, 25, 26, 27, 28, 29, 30, 35, 40, 45, 50, 55, 60, 65, 70, 80, 90, 100, 110, 120, 130, 140, 150, 160, 170, 180, 190, 200, 225, 250, 275, 300, 350, 400, 450, or 500 or more or any value or range of values therebetween in integer increments. In an exemplary embodiment, Z can equal 1, 2, 3, 4, 5, 6, 7, 8, 9, 10, 11, 12, 13, 14, 15, 16, 17, 19, 20, 21, 22, 23, 24, 25, 26, 27, 28, 29, 30, 35, 40, 45, 50, 55, 60, 65, 70, 80, 90, 100, 110, 120, 130, 140, 150, 160, 170, 180, 190, 200, 225, 250, 275, 300, 350, 400, 450, or 500 or more or any value or range of values therebetween in integer increments.

It is noted that the aforementioned “collective actions” can further include any one or more the method actions detailed herein to create a new collective action. It is also noted that these collective actions need not necessarily be contiguous with one another. By way of example only and not by way of limitation, in between collective action number 11 and collective action number 12, a fitting process that is based entirely on testing and having nothing to do with utilizing captured speech can take place. Indeed, in an exemplary embodiment, there can be P number of actions, or no more than P number of actions or at least P number of actions that include fitting the hearing prostheses that specifically do not include utilizing captured speech according to the teachings detailed herein, where P is 1, 2, 3, 4, 5, 6, 7, 8, 9, 10, 11, 12, 13, 14, 15, 16, 17, 18, 19, 20, 21, 22, 23, 24, or 25 or more or any value or range of values therebetween in one integer increments.

It is further noted that any of the methods detailed herein can be executed within any of the temporal periods detailed herein (e.g., 9000 hours).

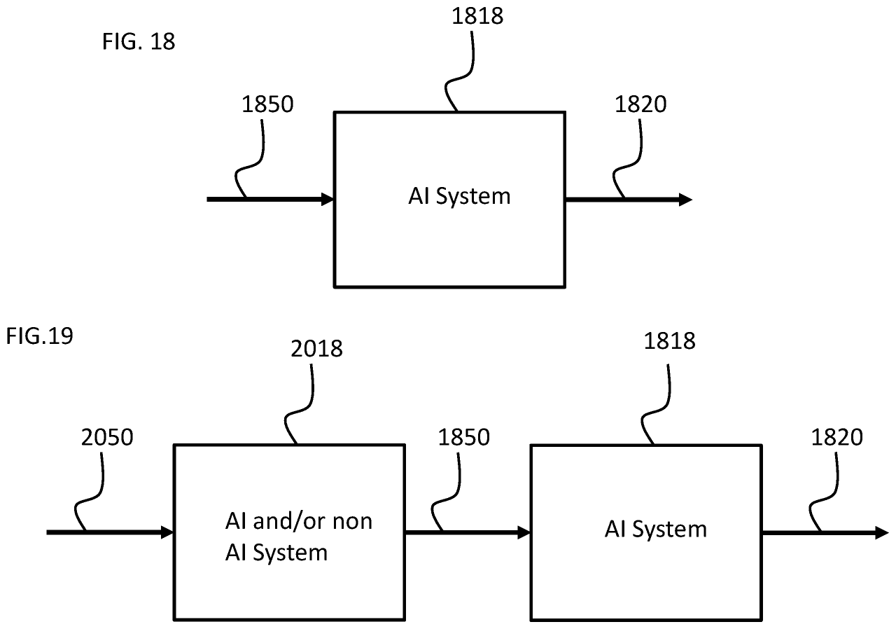

An exemplary embodiment includes taking an artificial intelligence-based analysis system that is configured to develop fitting data or otherwise analyze or provide a summary or report based on input indicative of a recipient's ability to hear and modifying such or otherwise constructing a system to work with such. In this regard, in an exemplary embodiment, there is an artificial intelligence-based analysis system that is configured to receive input indicative of recipient performance on one or more of the following types of tests: audiogram testing/tests to develop an audiogram; phoneme discrimination tests; loudness scaling tests and/or word tests. Beep tests can be used. (It is again noted that an embodiment can include using any of these tests/results of the tests, in conjunction with the analysis of the captured sound, to fit the hearing prosthesis or other prosthesis and/or to provide the summaries/reports herein. The system is configured to analyze this input utilizing an artificial intelligence-based processing system (e.g., expert system, neural network, etc.), and the system outputs recommended map adjustments or map settings or otherwise the system is utilized to fit a sensory prosthesis, such as a hearing prosthesis, to a recipient based on the input. Hereinafter, the system is referred to as system 1818, represented by black box 1818 in FIG. 18, where 1850 represents the input detailed above, and 1820 represents the output (fitting/map data, etc.).

In an exemplary embodiment, an interface is provided for system 1818 that is configured to take the speech data/sound data or otherwise captured sound that is captured according to the teachings detailed herein, analyze or otherwise manipulate that data, and develop an output that is compatible for use with system 1818. This system, which will be called system 2018, in an exemplary embodiment, can essentially operate to convert the data that is captured during the normal course of everyday life (the sound data) or otherwise extract information from that data and utilize the data to develop data that is analogous to the results of the aforementioned tests: audiogram testing/tests to develop an audiogram; phoneme discrimination tests; loudness scaling tests and/or word tests. By doing so, the output of system 2018 becomes compatible with utilization with system 1818. Thus, in an exemplary embodiment, system 2018 converts the sound data into test results data, even though no test has been given. FIG. 19 presents an exemplary embodiment of the utilization of system 2018 with system 1818, where the input 2050 can be any of the input detailed herein regarding the captured sound are captured speech (whether a raw signal or a processed signal or an abbreviated data set, or a representative data set, etc.) and can also include any of the data logging input detailed herein or variations thereof that will enable this conversion. The output of system 2018 is the input 1850.

In an exemplary embodiment, system 2018 is a processor-based system and/or an AI-based system, which can be an expert system, or a neural network, etc. Any system that can enable the functionality of system 2018 can be utilized in at least some exemplary embodiments. In an exemplary embodiment, input 2050 is provided to system 2018 in real time, while in other embodiments, input 2050 is provided to system 2018 periodically or whenever there is a utilitarian amount of data that is compiled. In an exemplary embodiment where the machine that is utilized to capture the sound or the like is a tape recorder or the like, every one or two or three or four or five days, etc., the recording of the sound that is captured can be inputted into the system 2018, thus constituting input 2050. Again, consistent with some embodiments, non-speech data can also be provided. In an exemplary embodiment, there is code of and/or from a machine learning algorithm in system 2018 (which can reside on a personal computer or otherwise can be a personal computer, and/or can be a smart phone or smart device or any of the devices disclosed herein, and can in some embodiments, be the hearing prosthesis or sensory prosthesis) which analyzes the input, and instead of developing the fitting data where the recommendations of the reports as detailed herein, instead analyzes the data to develop data that would correspond to any of the aforementioned tests. For example, system 2018 can analyze the data and develop pseudo audiograms based on the data, and thus create pseudo audiogram test results. In an exemplary embodiment, the system 2018 can analyze the input 2050 and develop pseudo phoneme test results. The system can analyze and put 2052 develop pseudo loudness scaling test results and/or word test results. The idea is that system 2018 analyzes the data and determines or otherwise estimates how the recipient would perform on any of the aforementioned tests based on the data, without giving the recipient the test.

This output 1850 can then be fed to system 1818 as if this was real test data, and system 1818 can do its thing as if the testator was real test data. In an exemplary embodiment, system 1818 never “knows” the difference.

It is noted that in an exemplary embodiment, system 1818 and system 2018 are subsystems in an overall system.

Accordingly, in an exemplary embodiment, there is a system that is configured to analyze a linguistic environment metric and convert the metric to pseudo-hearing test data, and to analyze the pseudo-hearing test data as if it was actual hearing test data to develop the fitting data.

At least some exemplary embodiments according to the teachings detailed herein utilize advanced learning signal processing techniques, which are able to be trained or otherwise are trained to detect higher order, and/or non-linear statistical properties of signals. Above, such was sometimes referred to as artificial intelligence. An exemplary signal processing technique is the so called deep neural network (DNN). At least some exemplary embodiments utilize a DNN (or any other advanced learning signal processing technique) to process a signal representative of captured sound, and, in other embodiments, other input (e.g., the results of the hearing test) as noted above. At least some exemplary embodiments entail training signal processing algorithms to process signals indicative of captured sound. That is, some exemplary methods utilize learning algorithms such as DNNs or any other algorithm that can have utilitarian value where that would otherwise enable the teachings detailed herein to analyze captured sound. It is noted that the aforementioned discussion focused on sound. It is noted that the teachings detailed herein can also be applicable to captured light. In this regard, the teachings detailed herein can be utilized to analyze or otherwise process a signal that is based on captured light, and evoke a sensory percept, such as a vision percept, based on the processed signal. Thus, in an exemplary embodiment, a neural network, such as a deep neural network (DNN) can be used to execute at least one or more of the method actions detailed herein. A so-called “product” of a DNN can be used. The product can be based on or be from a neural network. In an exemplary embodiment, the product is code. In an exemplary embodiment, the product is a logic circuit that is fabricated based on the results of machine learning. The product can be an ASIC (e.g., an artificial intelligence ASIC). The product can be implemented directly on a silicon structure or the like. Any device, system and or method that can enable the results of artificial intelligence to be utilized in accordance with the teachings detailed herein, such as in a hearing prosthesis or a component that is in communication with a hearing prosthesis, can be utilized in at least some exemplary embodiments. Indeed, as will be detailed below, in at least some exemplary embodiments, the teachings detailed herein utilize knowledge/information from an artificial intelligence system or otherwise from a machine learning system.

A “neural network” is a specific type of machine learning system. Any disclosure herein of the species “neural network” constitutes a disclosure of the genus of a “machine learning system.” Further, any disclosure herein of artificial intelligence corresponds to any one of the types of artificial intelligence detailed herein, and/or otherwise constitutes a disclosure of a neural network and/or a machine learning system, etc. While embodiments herein focus on the species of a neural network, it is noted that other embodiments can utilize other species of machine learning systems. Accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

To be clear, some embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network. The teachings detailed herein include utilizing machine learning methodologies and the like to establish sensory prosthetic devices or supplemental components utilized with sensory prostatic devices (e.g., a smart phone), to replace or otherwise augment the processing functions, etc. (e.g., sound or light processing, etc.) of a given sensory prostheses.

It is also noted that at least some exemplary embodiments utilize so-called expert systems as the artificial intelligence system. Any disclosure herein of a neural network or of a DNN and/or of an artificial intelligence system corresponds to a disclosure in an exemplary embodiment that utilizes an expert system providing that the art enables such, unless otherwise noted.

Some of the specifics of the DNN utilized in some embodiments will be described below, including some exemplary processes to train such DNN. First, however, some of the exemplary methods of utilizing such a DNN (or any other algorithm that can have utilitarian value) will be described.

As noted above, some methods entail processing the data utilizing a product of machine learning, such as the results of the utilization of a DNN, a machine learning algorithm or system, or any artificial intelligence system that can be utilized to enable the teachings detailed herein. This as contrasted from, for example, processing the data utilizing general code or utilizing code that not from a machine learning algorithm or utilizing a non AI based/resulting chip, etc. In an exemplary embodiment, a typical cochlear implant processes a signal from a microphone and subsequently provides the results of that processing to a stimulation device that stimulates various electrodes in a weighed manner. This processing is typically done by a sound processor which includes filter banks that simply divides up an input signal into separate filter groups or filter bins. This is not the utilization of a machine learning algorithm. That said, it is noted that in some embodiments, this division can be executed utilizing results from machine learning (e.g., a trained DNN, on whatever medium that can enable such, such as a chip).

Again, in an exemplary embodiment, the machine learning can be a DNN, and the product can correspond to a trained DNN and/or can be a product based on or from the DNN (more on this below). It is noted that in at least some exemplary embodiments, the DNN or the code from a machine learning algorithm, etc., is utilized to achieve a given functionality as detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or code from a machine learning algorithm, or an artificial intelligence system etc., that when used, results in that functionality, unless otherwise noted or unless the art does not enable such.

Any learning model that is available and can enable the teachings detailed herein can be utilized in at least some exemplary embodiments. As noted above, an exemplary model that can be utilized with voice analysis and other audio tasks is the Deep Neural Network (DNN). Again, other types of learning models can be utilized, but the following teachings will be focused on a DNN. At least some of the method actions detailed herein include processing data based on the audio and/or visual content using code from a machine learning algorithm to develop output. In an exemplary embodiment, this can correspond to processing the raw signal from the microphone, and thus the data based on the audio and/or visual content is the data that is obtained in at least some exemplary methods detailed herein or otherwise via the input output subsystems, etc. As noted above, at least some exemplary method actions detailed herein entail processing the data utilizing code from a machine learning algorithm. This as contrasted from, for example, processing the data utilizing code that not from a machine learning algorithm. Again, in an exemplary embodiment, the machine learning algorithm can be a DNN, and the code can correspond to a trained DNN and/or can be a code from the DNN (more on this below).

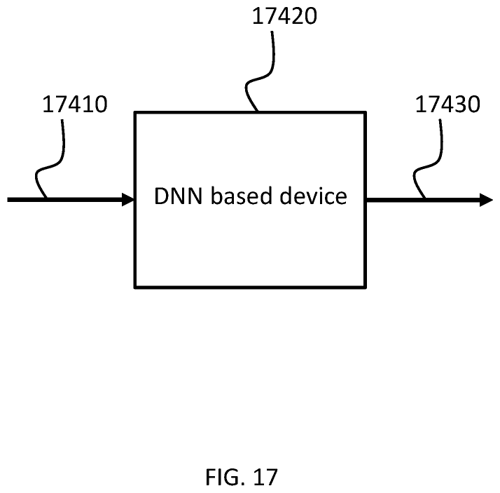

FIG. 17 depicts an exemplary conceptual functional black box schematic associated with the method actions detailed above, where a sound signal 17410 is the input into a DNN based device 17420 that utilizes a trained DNN or some other trained learning algorithm (or the results thereof—the code of a machine learning algorithm as used herein corresponds to a trained learning algorithm as used in operational mode after training has ceased and code from a machine learning algorithm corresponds to a code that is developed as a result of training of the algorithm—again, this will be described in greater detail below), and the output 17430 can be the evaluation of the report or the fitting data, etc., detailed above, and the output 17430 can be directed to a cochlear implant or other type of hearing prostheses that is fitted based on that output. In this exemplary embodiment, device 17420 can be smart phone or a personal computer or a mainframe computer where the cochlear implant or other implant.

It is noted that in at least some exemplary embodiments, the input 17410 comes directly from a microphone, while in other embodiments, this is not the case. Input 17410 can correspond to any input that can enable the teachings detailed herein to be practiced providing that the art enables such. Thus, in some embodiments, there is no “raw sound” input into the DNN. Instead, it is all pre-processed data. Any data that can enable the DNN or other machine learning algorithm to operate can be utilized in at least some exemplary embodiments.

Some additional features of the device 17420 are described above. It is noted that at least some embodiments can include methods, devices, and/or systems that utilize a DNN inside a cochlear implant system, middle ear implant system, bone conduction implant system (or non-implant), a conventional hearing aid and/or a personal hearing device (e.g., a headset attached to a smartphone or the like, where the microphone of the smart phone is utilized to capture sound, and the smart phone amplifies the sound and provides it to the headset for the benefits of the recipient) or a sight prosthesis, such as a retinal implant raid bionic eye, etc., and/or along with such a system. The neural network can be, in some embodiments, either a standard pre-trained network where weights have been previously determined (e.g., optimized) and loaded onto the network, or alternatively, the network can be initially a standard network, but is then trained to improve specific recipient results based on outcome oriented reinforcement learning techniques.

According to an exemplary embodiment of developing a learning model, a learning model type is selected and structured, and the features and other inputs are decided upon and then the system is trained. It needs to be trained. In exemplary embodiments of training the system, a utilitarian amount of real data is compiled and provided to the system. In an exemplary embodiment, the real data comprises any data having utilitarian value. The learning system then changes its internal workings and calculations to make its own estimation closer to, for example, the actual person's hearing outcome. This internal updating of the model during the training phase can improve (and should improve) the system's ability to correctly control the prosthesis. Subsequent individual subject's inputs and outputs are presented to the system to further refine the model. With training according to such a regime, the model's accuracy is improved. In at least some exemplary embodiments, the larger and broader the training set, the more accurate the model becomes. In the case of a DNN, the size of the training can depend on the number of neurons in the input layer, hidden layer(s), and output layer.

There are many packages now available to perform the process of training the model. Simplistically, the input measures are provided to the model. Then the outcome is estimated. This is compared to the subject's actual outcome, and an error value is calculated. Then the reverse process is performed using the actual subject's outcome and their scaled estimation error to propagate backwards through the model and adjust the weights between neurons, and improving its accuracy (hopefully). Then a new subject's data is applied to the updated mode, providing a (hopefully) improved estimate. This is simplistic, as there are a number of parameters apart from the weight between neurons which can be changed, but generally shows the typical error estimation and weight changing methods for tuning models according to an exemplary embodiment.

A system utilized to train a DNN or any other machine learning algorithm, along with acts associated therewith, is now described. Again, consistent with the statements detailed above, a DNN is utilized as but one example. Embodiments include utilizing the teachings detailed herein with respect to an expert system or any other type of artificial intelligence system that can have utilitarian value. Again, consistent with the statements detailed above, any disclosure below of a DNN corresponds to a disclosure of an embodiment of another type of artificial intelligence system, such as an expert system, disclosed herein.

The system will be described, at least in part, in terms of interaction with a recipient, although that term is used as a proxy for any pertinent subject to which the system is applicable (e.g., the test subjects used to train the DNN, the subject utilized to validate the trained DNN.). In an exemplary embodiment, system 1206, as seen in FIG. 15, is a recipient-controlled system while in other embodiments, it is a remote-controlled system. In an exemplary embodiment, system 1206 can correspond to a remote device and/or system, which, as detailed above, can be a portable handheld device (e.g., a smart device, such as a smart phone), and/or can be a personal computer, etc. In an exemplary embodiment, the system is under the control of an audiologist or the like, and subjects visit an audiologist center.

In an exemplary embodiment, the system can be a system having additional functionality according to the method actions detailed herein. In the embodiment illustrated in FIG. 16, the device 100 can be connected to system 1206 to establish a data communication link 1208 between the hearing prosthesis 100 (where hearing prosthesis 100 is a proxy for any device that can enable the teachings detailed herein, such as a smartphone with a microphone, a dedicated microphone, a phone, etc.) and system 1206. System 1206 is thereafter bi-directionally coupled by a data communication link 1208 with hearing prosthesis 100. Any communications link that will enable the teachings detailed herein that will communicably couple the implant and system can be utilized in at least some embodiments.

System 1206 can comprise a system controller 1212 as well as a user interface 1214. Controller 1212 can be any type of device capable of executing instructions such as, for example, a general or special purpose computer, a handheld computer (e.g., personal digital assistant (PDA)), digital electronic circuitry, integrated circuitry, specially designed ASICs (application specific integrated circuits), firmware, software, and/or combinations thereof. As will be detailed below, in an exemplary embodiment, controller 1212 is a processor. Controller 1212 can further comprise an interface for establishing the data communications link 1208 with the hearing prosthesis 100 (again, which is a proxy for any device that can enable the methods herein—any device with a microphone and/or with an input suite that permits the input data for the methods herein to be captured). In embodiments in which controller 1212 comprises a computer, this interface may be, for example, internal or external to the computer. For example, in an exemplary embodiment, controller 1206 and cochlear implant may each comprise a USB, FireWire, Bluetooth, Wi-Fi, or other communications interface through which data communications link 1208 may be established. Controller 1212 can further comprise a storage device for use in storing information. This storage device can be, for example, volatile or non-volatile storage, such as, for example, random access memory, solid state storage, magnetic storage, holographic storage, etc.

In an exemplary embodiment, input 1000 is provided into system 1206. The DNN signal analysis device 1020 analyzes the input 1000, and provides output 1040 to model section 1050, which establishes the model that will be utilized for the trained device. The output 1060 is thus the trained neural network, which is then uploaded onto the prostheses or the smartphone or other component that is utilized to implement the trained neural network.

Here, the neural network can be “fed” statistically significant amounts of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. In one exemplary embodiment, a machine learning algorithm starts off untrained, and then the machine learning algorithm is trained and “graduates” or matures into a usable code—code of trained machine learning algorithm. With respect to another exemplary embodiment, the code from a trained machine learning algorithm—is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice of the first path, thus are not present in the first version. Instead, only the resulting product of the learning is used.

In an exemplary embodiment, the code from and/or of the machine learning algorithm utilizes non-heuristic processing to develop the data utilized in the trained system. In this regard, the system takes sound data or takes in general relating to sound, and extracts fundamental signal(s) there from, and uses this to develop the model. By way of example only and not by way of limitation, the system utilizes algorithms beyond a first-order linear algorithm, and “looks” at more than a single extracted feature. Instead, the algorithm “looks” to a plurality of features. Moreover, the algorithm utilizes a higher order nonlinear statistical model, which self learns what feature(s) in the input is important to investigate. As noted above, in an exemplary embodiment, a DNN is utilized to achieve such. Indeed, in an exemplary embodiment, as a basis for implementing the teachings detailed herein, there is an underlying assumption that the features of the sound and other input into the system that enable the model to be generated may be too complex to be specified, and the DNN is utilized in a manner without knowledge as to what exactly on which the algorithm is basing its determinations/at which the algorithm is looking to develop the model.

In at least some exemplary embodiments, the DNN is the resulting code used to make the prediction. In the training phase there are many training operations algorithms which are used, which are removed once the DNN is trained.

To be clear, in at least some exemplary embodiments, the trained algorithm is such that one cannot analyze the trained algorithm with the resulting code there from to identify what signal features or otherwise what input features are utilized to produce the output of the trained neural network. In this regard, in the development of the system, the training of the algorithm, he system is allowed to find what is most important on its own based on statistically significant data provided thereto. In some embodiments, it is never known what the system has identified as important at the time that the system's training is complete. The system is permitted to work itself out to train itself and otherwise learn to control the prosthesis.

Briefly, it is noted that at least some of the neural networks or other machine learning algorithms utilized herein do not utilize correlation, or, in some embodiments, do not utilize simple correlation, but instead develop relationships. In this regard, the learning model is based on utilizing underlying relationships which may not be apparent or otherwise even identifiable in the greater scheme of things. In an exemplary embodiment, MatLAB, Buildo, etc., are utilized to develop the neural network. In some exemplary embodiments detailed herein, the resulting train system is one that is not focused on a specific speech feature, but instead is based on overall relationships present in the underlying statistically significant samples provided to the system during the learning process. The system itself works out the relationships, and there is no known correlation based on the features associated with the relationships worked out by the system.

The end result is a code which is agnostic to sound features. That is, the code of the trained neural network and/or the code from the trained neural network is such that one cannot identify what sound features are utilized by the code to develop the production (the output of the system). The resulting arrangement is a complex arrangement of an unknown number of features of sound that are utilized. The code is written in the language of a neural network, and would be understood by one of ordinary skill in the art to be such, as differentiated from a code that utilized specific and known features. That is, in an exemplary embodiment, the code looks like a neural network.

Consistent with common neural networks, there are hidden layers, and the features of the hidden layer are utilized in the process to predict the hearing impediments of the subject.

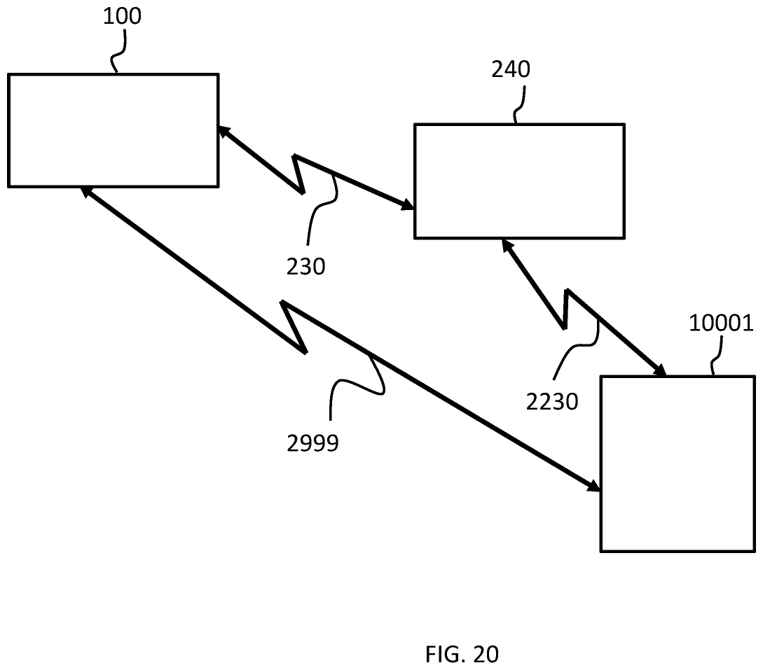

FIG. 20 depicts an exemplary functional schematic, where the remote device 240 is in communication with a geographically remote device/facility 10001 via link 2230, which can be an internet link. The geographically remote device/facility 10001 can encompass controller 1212, and the remote device 240 can encompass the user interface 1214. Also, as can be seen, there can be a direct link 2999 with the prosthesis 100 and the remote facility 10001

Accordingly, an exemplary embodiment entails executing some or all of the method actions detailed herein where the recipient of the hearing prosthesis, the hearing prosthesis 100 and/or the portable handheld device 240 is located remotely (e.g., geographically distant) from where at least some of the method actions detailed herein are executed.

In view of the above, it can be seen that in an exemplary embodiment, there is a portable handheld device, such as portable handheld device 240, comprising a cellular telephone communication suite (e.g., the phone architecture of a smartphone), and a hearing prosthesis functionality suite, (e.g., an application located on the architecture of a smartphone that enables applications to be executed that is directed towards the functionality of a hearing prosthesis) including a touchscreen display. In an exemplary embodiment, the hearing prosthesis functionality suite is configured to enable a recipient to adjust a feature of a hearing prosthesis, such as hearing prosthesis 100, remote from the portable handheld device 240 via the touchscreen display (e.g., by sending a signal via link 230 to the hearing prosthesis 100).

It is noted that in describing various teachings herein, various actions and/or capabilities have been attributed to various elements of the system 210. In this regard, any disclosure herein associated with a given functionality or capability of the hearing prosthesis 100 also corresponds to a disclosure of a remote device 240 (e.g., a portable handheld device) having that given functionality or capability providing that the art enables such and/or a disclosure of a geographically remote facility 10001 having that given functionality or capability providing that the art enables such. Corollary to this is that any disclosure herein associated with a given functionality or capability of the remote device 240 also corresponds to a disclosure of a hearing prosthesis 100 having that given functionality or capability providing that the art enables such and/or disclosure of a geographically remote facility 10001 having that given functionality or capability, again providing that the art enables such. As noted above, the system 210 can include the hearing prosthesis 100, the remote device 240, and the geographically remote device 1000.

It is further noted that the data upon which determinations are made or otherwise based with respect to the display of a given interface display can also correspond to data relating to a more generalized use of the system 210. In this regard, in some embodiments, the remote device 240 and/or the hearing prosthesis 100 can have a so-called caregiver mode, where the controls or data that is displayed can be more sophisticated relative to that which is the case for the normal control mode/the recipient control mode. By way of example only and not by way of limitation, if the recipient is a child or one having diminished faculties owing to age or ailment, the system 210 can have two or more modes. Accordingly, the data detailed herein can corresponds to input regarding which mode the system 210 is being operated in, and a given display can be presented based on that mode. For example, the caregiver display can have more sophisticated functionalities and/or the ability to adjust more features and/or present more data than the recipient mode. In an exemplary embodiment, a user can input into the remote device 240 a command indicating that the hearing prosthesis is to be operated in caregiver mode, and the displays presented thereafter caregiver mode displays, and these displays are presented until a command is entered indicating that the hearing prosthesis is to be operated in recipient mode, after which displays related to recipient mode are displayed (until a caregiver command is entered, etc.). That said, in an alternate embodiment, a caregiver and/or the recipient need not enter specific commands into system 210. In an exemplary embodiment, system 210 is configured to determine what mode it should be operated in. By way of example only and not by way of limitation, if a determination is made that the caregiver's voice has been received within a certain temporal period by the hearing prosthesis 100, the system 210 can enter the caregiver mode and present the given displays accordingly (where if the caregiver's voice is not been heard within a given period of time, the default is to a recipient control mode). Corollary to this is that in at least some exemplary embodiments, two or more remote devices 240 can be utilized in system 210, one of which is in the possession of the recipient, and another of which is in the possession of the caregiver. Depending on the data, various displays are presented for the various remote devices 240.

It is briefly noted that in an exemplary embodiment, as was described above, the cochlear implant 100 and/or the device 240 is utilized to capture speech/voice of the recipient and/or people speaking to the recipient. Further, as was described above, the implant 100 and/or the device 240 can be used to log data, which data can be non-speech and/or non-voice based data relating to the use of the implant by a recipient thereof, such as, by way of example only and not by way of limitation, coil on/coil off time, etc. It is briefly noted that any disclosure herein of voice (e.g., capturing voice, analyzing voice, etc.) corresponds to a disclosure of an alternate embodiment of using speech (e.g., capturing speech, analyzing speech, etc.), and vice versa, unless otherwise specified, providing that the art enables such. This is not to say that the two are synonymous. This is to say that in the interests of textual economy, we are presenting multiple disclosures based on the use of one. It is also noted that in at least some instances herein, the phrase voice sound is used. This corresponds to the sound of one's voice, and can also be referred to as “voice.”

In an exemplary embodiment, own voice detection is executed according to any one or more of the teachings of U.S. Patent No. 2016/0080878 and/or the implementation of the teachings associated with the detection of the invoice herein are executed in a manner that triggers the control techniques of that application. Accordingly, in at least some exemplary embodiments, the prosthesis 100 and/or the device 240 and/or the remote device are configured to or otherwise include structure to execute one or more or all of the actions detailed in that patent application. Moreover, embodiments include executing methods that correspond to the execution of one or more the method actions detailed in that patent application.

In an exemplary embodiment, own voice detection is executed according to any one or more of the teachings of WO 2015/132692 and/or the implementation of the teachings associated with the detection of the invoice herein are executed in a manner that triggers the control techniques of that application. Accordingly, in at least some exemplary embodiments, the prosthesis 100 and/or the device 240 and/or the remote device are configured to or otherwise include structure to execute one or more or all of the actions detailed in that patent application. Moreover, embodiments include executing methods that correspond to the execution of one or more the method actions detailed in that patent application.

An exemplary embodiment includes capturing the voice of a recipient of the prosthesis detailed herein and/or the voice of a hearing-impaired person in a conversational manner/where the recipient with a person of interest is talking in a conversational tone.

Again, exemplary embodiments include any device and/or system that can enable the capturing of ambient sound in general, and speech sounds in particular, that are around or otherwise to which the recipient is exposed. In at least some exemplary embodiments, there are method actions that include capturing speech/voice sounds sound with a machine, such as, for example, implant 100 and/or device 240 detailed above, or the system 210. In an exemplary embodiment, the captured voice can be captured by the microphone of the implant 100. In an exemplary embodiment, the voice can be recorded and stored in the implant 100 and/or in a component associated with the system 210 and/or can be uploaded via element 249 in real time or impartial real time. A simple tape recorder is utilized to execute the action of capturing speech. In an alternate embodiment, a laptop computer is utilized, which can utility with respect to someone who works in an office or the like. It is noted that in at least some exemplary embodiments, subsequent to capturing the sound, there is an action at of analyzing or otherwise reducing the captured voice to data indicative of the captured voice and/or data indicative of one or more properties of the captured voice, which data then can be stored in the implant of the system and/or stored in whatever device captured the sound and/or is communicated to a remote server, etc., to implement the teachings detailed herein. Some embodiments utilize data that is a distillation of the captured sound to execute the teachings detailed herein, as opposed to the total captured sound. By way of example only and not by way of limitation, a captured sound that include the voice of the recipient and the person to whom he or she is speaking, as well as others in the background, might be manipulated or otherwise reduced to eliminate the sound of the others in the background if such are not pertinent to evaluating the recipient's ability to hear. Further, in an exemplary embodiment, frequencies outside of voice range may be eliminated, thus reducing the size of the data. Thus, by “based on captured sound,” such includes both the complete audio signal, as well as a manipulated portion of that audio signal.

As noted above, at least some exemplary embodiments also include logging data with a machine, which can be the machine that was utilized to capture the sound, and/or can be another machine. In an exemplary embodiment, the logged data is non-voice based data corresponding to events and/or actions in a recipient of a hearing prosthesis's real world auditory environment, wherein the recipient is a person who spoke the captured voice and/or to whom the captured voice was spoken. In an embodiment, the data relates to the use of a hearing prosthesis by a recipient who spoke the captured voice and/or to whom the captured voice was spoken.

An alternate embodiment includes a method, comprising capturing an individual's voice with a machine and logging data corresponding to events and/or actions of the individual's real world auditory environment, wherein the individual is speaking while using a hearing assistance device, and the hearing assistance device at least one of corresponds to the machine or is a device used to execute the action of logging data.

In at least some exemplary embodiments, systematic methods of evaluating the voice sound using natural language processing (NLP) can be utilized.

In at least some exemplary embodiments, linguistic features that are associated with spoken text, based on empirical results from studies, for example, are utilized in at least some exemplary embodiments, to evaluate the voice sound. At least some algorithms utilize one or two or three or four or five dimensional measurements.

It is explicitly noted that at least some exemplary embodiments include the teachings herein when combined with the non-voice data logging detailed herein and/or the scene classification logging detailed herein. When used in combination, such can be directed towards identifying a weakness in a recipient's map.

It is further explicitly noted that at least some exemplary embodiments include the teachings herein without the aforementioned data logging. Here however, the voice is evaluated to determine features associated with the higher levels of hearing.

In some embodiments, an integrated or plug-in microphone is coupled to an optional pre-processing component that can provide a variety of functions such as A/D conversion, digital/analog filtering, compression, automatic gain control, balance, noise reduction, and the like. The preprocessed signal is coupled to a processor component that works cooperatively with memory to execute programmed instructions. Optionally, mass storage may be provided in the device itself as has become available in media player devices such as the iPod produced by Apple Computer, Inc. Alternatively, mass storage may be omitted, which would prohibit the use of logging or subsequent analysis, or mass storage may be implemented remotely via devices coupled to the external input/output. The user interface may be implemented as a graphical, text only, or hardware display depending on the level of information required by a user.

In at least some exemplary embodiments of the teachings detailed herein, signals are detected by the microphone, pre-processed if necessary or desired, and provided as input to the processing component. In one embodiment, the processor component functions to store pre-processed voice signals in memory and/or mass storage for subsequent, asynchronous analysis. Further by example, a predefined word or phrase list is loaded into memory where each word is represented by text and/or each word is represented as a digital code that more readily matched to the pre-processed voice signal that is presented to the processor component.

Alternatively, or in addition, the room in which the communication occurs can be outfitted with one or more microphones that are coupled to computer system via wired (e.g., universal serial bus or sound card connection) or wireless connections.

A computer system may be implemented as a personal computer, laptop computer, workstation, handheld computer or special-purpose appliance specifically designed to implement some teachings herein. It is contemplated that some or all of the voice analysis functionality may be implemented in a wearable computer and/or integrated with voice capture device, or provided in a device such as a dictation machine, cell phone, voice recorder, MP3 recorder/player, iPod by Apple Computers Inc., or similar device.

It is noted that any method detailed herein also corresponds to a disclosure of a device and/or system configured to execute one or more or all of the method actions associated there with detailed herein. In an exemplary embodiment, this device and/or system is configured to execute one or more or all of the method actions in an automated fashion.

It is noted that embodiments include non-transitory computer-readable media having recorded thereon, a computer program for executing one or more or any of the method actions detailed herein. Indeed, in an exemplary embodiment, there is a non-transitory computer-readable media having recorded thereon, a computer program for executing at least a portion of any method action detailed herein.

Any action disclosed herein that is executed by the prosthesis 100 can be executed by the device 240 and/or the remote system in an alternative embodiment, unless otherwise noted or unless the art does not enable such. Thus, any functionality of the prosthesis 100 can be present in the device 240 and/or the remote system an alternative embodiment. Thus, any disclosure of a functionality of the prosthesis 100 corresponds to structure of the device 240 and/or the remote system that is configured to execute that functionality or otherwise have a functionality or otherwise to execute that method action.

Any action disclosed herein that is executed by the device 240 can be executed by the prosthesis 100 and/or the remote system in an alternative embodiment, unless otherwise noted or unless the art does not enable such. Thus, any functionality of the device 240 can be present in the prosthesis 100 and/or the remote system an alternative embodiment. Thus, any disclosure of a functionality of the device 240 corresponds to structure of the prosthesis 100 and/or the remote system that is configured to execute that functionality or otherwise have a functionality or otherwise to execute that method action.

Any action disclosed herein that is executed by the remote system can be executed by the device 240 and/or the prosthesis 100 in an alternative embodiment, unless otherwise noted or unless the art does not enable such. Thus, any functionality of the remote system can be present in the device 240 and/or the prosthesis 100 as alternative embodiment. Thus, any disclosure of a functionality of the remote system corresponds to structure of the device 240 and/or the prosthesis 100 that is configured to execute that functionality or otherwise have a functionality or otherwise to execute that method action. It is further noted that any disclosure of a device and/or system detailed herein also corresponds to a disclosure of otherwise providing that device and/or system. It is also noted that any disclosure herein of any process of manufacturing other providing a device corresponds to a device and/or system that results there from. Is also noted that any disclosure herein of any device and/or system corresponds to a disclosure of a method of producing or otherwise providing or otherwise making such. Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion. While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A fitting system, comprising:
a communications subsystem including at least one of an input subsystem and an output subsystem or an input/output subsystem; and
a processing subsystem, wherein
the processing subsystem is configured to:
automatically develop fitting data for a hearing prosthesis at least partially based on data inputted via the communications subsystem.

2. The fitting system of claim 1, wherein at least one of:
the fitting system is configured to develop the fitting data for the hearing prosthesis by analyzing a linguistic environment metric inputted into the communications subsystem; or
the fitting system is configured to develop the fitting data for the hearing prosthesis by analyzing a linguistic environment metric inputted into the communications subsystem and a non-listening metric inputted into the communications subsystem or another subsystem.

3. The fitting system of claim 2, wherein:
the fitting system is configured to develop the fitting data for the hearing prosthesis by analyzing the linguistic environment metric inputted into the communications subsystem; and
the system includes a sub-system including at least one of the hearing prosthesis or a portable body carried electronic device, the hearing prosthesis is configured to output data indicative of a linguistic environment of the recipient and the portable electronic device is configured to receive data indicative of the linguistic environment of the recipient; and
the linguistic environment metric is based on at least one outputted data or the received data.

4. The fitting system of claim 3, wherein:
the sub-system includes the portable electronic device;
the portable electronic device is a smart device, and
the processing subsystem is at least in part located in the smart device.

5. The fitting system of claim 2, wherein:
the processing subsystem is an expert sub-system that includes factual domain knowledge and clinical experience of experts as heuristics; and
the expert sub-system is configured to automatically develop the fitting data based on the metric.

6-7. (canceled)

8. The fitting system of claim 1, wherein:
the fitting system is configured to automatically develop the fitting data based effectively on passive error identification.

9. (canceled)

10. The fitting system of claim 2, wherein:
the system is configured to automatically develop revised fitting data for the hearing prosthesis based on subjective preference input from the recipient about the developed fitting data.

11-24. (canceled)

25. A non-transitory computer-readable media having recorded thereon, a computer program for executing at least a portion of a hearing-prosthesis fitting method, the computer program including:
code for enabling a obtaining of first data indicative of a speech environment of the recipient;
code for analyzing the obtained first data; and
code for developing fitting data based on the analyzed first data.

26. The media of claim 25, wherein:
the media is for a self-fitting method for the hearing prosthesis that enables a recipient thereof to self-fit the hearing prosthesis.

27. The media of claim 25, wherein:
the media is for an automatic fitting method that enables the automatic fitting of the hearing prosthesis based on the speech environment.

28. The media of claim 26, further comprising:
code for operating a system executing at least a part of the method in a fitting test mode;
code for enabling a obtaining second data indicative of a recipient of the hearing prostheses' perception of fitting test auditory information obtained while operating in the fitting test mode; and
code for analyzing the obtained second data, wherein
the code for developing the fitting data is also code for developing such based on the analyzed second data.

29. The media of claim 28, wherein:
the code for the enabling of the obtaining of the second data enables a system executing at least part of the method to obtain the second data via active activity on the part of the recipient of the hearing prosthesis.

30. (canceled)

31. The media of claim 26, wherein:
the code for analyzing is based on artificial intelligence.

32-39. (canceled)

40. A device, comprising:
a processor; and
a memory, wherein
the device is configured to receive input indicative of speech sound,
the device is configured to analyze the input indicative of speech sound, and
the device is configured to identify anomalies in the speech sound based on the analysis of the input, which anomalies are statistically related to hearing prosthesis fitting imperfections.

41. The device of claim 40, wherein:
the device is configured to analyze the identified anomalies and differentiate between anomalies that are indicative of a hearing problem from those that are not indicative of a hearing problem.

42. (canceled)

43. The device of claim 40, wherein:
the device is configured to identify the occurrence of repeated errors with respect to discrimination between specific phonemes as part of the analysis of the input and identify such as anomalies; and
the device is configured to develop fitting data for the hearing prosthesis based on the identified repeated errors.

44. The device of claim 43, wherein:
the device is configured to develop the fitting data based on data comprising fitting settings for hearing prostheses that have alleviated the errors for a statistically significant pool of people.

45. (canceled)

46. The device of claim 40, wherein:
the device is configured to automatically fit and/or refit a hearing prosthesis based solely on the identified anomalies.

47. The device of claim 40, wherein:
the device enables performance-based fitting of a hearing prosthesis.

48. The device of claim 40, wherein:
the device includes code of and/or from a machine learning algorithm that is used by the processor to identify the anomalies in the speech sound.

49. (canceled)
